SEQUENCE LISTING (20 sequences)

SEQ ID NO: 1 - protein, 112 aa, Human
GPELVKPGASVKISCKASGYAFSYSWMNWVKLRPGQGLEWIGRIFPGDGDTDYNGKFKGKATLTADKSSNTAYMQLTSLTSVDSAVYLCARNVFDGYWLVYWGQGTLVTVSA

SEQ ID NO: 2 - protein, 103 aa, Human
NPVTLGTSASISCRSSKSLLHSNGITYLYWYLQKPGQSPQLLIYQMSNLVSGVPDRFSSSGSGTDFTLRISRVEAEDVGVYYCAQNLELPYTFGGGTKLEIKR

SEQ ID NO: 3 - protein, 119 aa, Human
QVQLVQSGAEVKKPGSSVKVSCKASGYAFSYSWMNWVRQAPGQGLEWMGRIFPGDGDTDYNGKFKGRVTITADKSTSTAYMELSSLRSEDTAVYYCARNVFDGYWLVYWGQGTLVTVSS

SEQ ID NO: 4 - protein, 119 aa, Human
QVQLVQSGAEVKKPGSSVKVSCKASGYAFSYSWMNWVRQAPGQGLEWMGRIFPGDGDTDYNGKFKGRVTITADKSTSTAYMELSSLRSEDTAVYLCARNVFDGYWLVYWGQGTLVTVSS

SEQ ID NO: 5 - protein, 119 aa, Human
QVQLVQSGAEVKKPGASVKVSCKVSGYAFSYSWMNWVRQAPGQGLEWMGRIFPGDGDTDYNGKFKGRVTITADKSTSTAYMELSSLRSEDTAVYYCARNVFDGYWLVYWGQGTLVTVSS

SEQ ID NO: 6 - protein, 119 aa, Human
QVQLVQSGAEVKKPGSSVKVSCKASGYAFSYSWMSWVRQAPGQGLEWMGRIFPGDGDTDYNGKFKGRVTITADKSTSTAYMELSSLRSEDTAVYYCARNVFDGYWLVYWGQGTLVTVSS

SEQ ID NO: 7 - protein, 119 aa, Human
QVQLVQSGAEVKKPGSSVKVSCKASGYAFSYSWINWVRQAPGQGLEWMGRIFPGDGDTDYNGKFKGRVTITADKSTSTAYMELSSLRSEDTAVYYCARNVFDGYWLVYWGQGTLVTVSS

SEQ ID NO: 8 - protein, 119 aa, Human
QVQLVQSGAEVKKPGSSVKVSCKASGYAFSYSWISWVRQAPGQGLEWMGRIFPGDGDTDYNGKFKGRVTITADKSTSTAYMELSSLRSEDTAVYYCARNVFDGYWLVYWGQGTLVTVSS

SEQ ID NO: 9 - protein, 119 aa, Human
QVQLVQSGAEVKKPGASVKVSCKASGYTFTYSWMNWVRQAPGQGLEWMGRIFPGDGDTDYNGKFKGRVTITADKSTSTAYMELSSLRSEDTAVYYCARNVFDGYWLVYWGQGTLVTVSS

SEQ ID NO: 10 - protein, 119 aa, Human
QVQLVQSGAEVKKPGASVKVSCKASGYTFSYSWMNWVRQAPGQGLEWMGRIFPGDGDTDYNGKFKGRVTITADKSTSTAYMELSSLRSEDTAVYYCARNVFDGYWLVYWGQGTLVTVSS

SEQ ID NO: 11 - protein, 119 aa, Human
EVQLVESGGGLVKPGGSLRLSCAASGFTFSYSWMNWVRQAPGKGLEWVGRIFPGDGDTDYNGKFKGRVTITADKSTSTAYMELSSLRSEDTAVYYCARNVFDGYWLVYWGQGTLVTVSS

SEQ ID NO: 12 - protein, 119 aa, Human
EVQLVESGGGLVKPGGSLRLSCAASGFAFSYSWMNWVRQAPGKGLEWVGRIFPGDGDTDYNGKFKGRVTITADKSTSTAYMELSSLRSEDTAVYYCARNVFDGYWLVYWGQGTLVTVSS

SEQ ID NO: 13 - protein, 119 aa, Human
QVQLVESGGGLVKPGGSLRLSCAASGFTFSYSWMNWVRQAPGKGLEWVGRIFPGDGDTDYNGKFKGRVTITADKSTSTAYMELSSLRSEDTAVYYCARNVFDGYWLVYWGQGTLVTVSS

SEQ ID NO: 14 - protein, 119 aa, Human
EVQLVESGAGLVKPGGSLRLSCAASGFTFSYSWMNWVRQAPGKGLEWMGRIFPGDGDTDYNGKFKGRVTITADKSTSTAYMELSSLRSEDTAVYYCARNVFDGYWLVYWGQGTLVTVSS

SEQ ID NO: 15 - protein, 119 aa, Human
EVQLVESGGGVVKPGGSLRLSCAASGFTFSYSWMNWVRQAPGKGLEWMGRIFPGDGDTDYNGKFKGRVTITADKSTSTAYMELSSLRSEDTAVYYCARNVFDGYWLVYWGQGTLVTVSS

SEQ ID NO: 16 - protein, 119 aa, Human
EVQLVESGGGLKKPGGSLRLSCAASGFTFSYSWMNWVRQAPGKGLEWMGRIFPGDGDTDYNGKFKGRVTITADKSTSTAYMELSSLRSEDTAVYYCARNVFDGYWLVYWGQGTLVTVSS

SEQ ID NO: 17 - protein, 119 aa, Human
EVQLVESGGGLVKPGSSLRLSCAASGFTFSYSWMNWVRQAPGKGLEWMGRIFPGDGDTDYNGKFKGRVTITADKSTSTAYMELSSLRSEDTAVYYCARNVFDGYWLVYWGQGTLVTVSS

SEQ ID NO: 18 - protein, 119 aa, Human
EVQLVESGGGLVKPGGSLRVSCAASGFTFSYSWMNWVRQAPGKGLEWMGRIFPGDGDTDYNGKFKGRVTITADKSTSTAYMELSSLRSEDTAVYYCARNVFDGYWLVYWGQGTLVTVSS

SEQ ID NO: 19 - protein, 119 aa, Human
EVQLVESGGGLVKPGGSLRLSCAASGFTFSYSWMNWVRQAPGKGLEWMGRIFPGDGDTDYNGKFKGRVTITADKSTSTAYMELSSLRSEDTAVYYCARNVFDGYWLVYWGQGTLVTVSS

SEQ ID NO: 20 - protein, 115 aa, Human
DIVMTQTPLSLPVTPGEPASISCRSSKSLLHSNGITYLYWYLQKPGQSPQLLIYQMSNLVSGVPDRFSGSGSGTDFTLKISRVEAEDVGVYYCAQNLELPYTFGGGTKVEIKRTV

(19) United States
(12) Patent Application Publication
(10) Pub. No.: US 2020/0237905 A1
(43) Pub. Date: Jul. 30, 2020

(54) COMBINATION THERAPY WITH A BET INHIBITOR, A BCL-2 INHIBITOR AND AN ANTI-CD20 ANTIBODY

(71) Applicant: Hoffmann-La Roche Inc., Little Falls, NJ (US)

(72) Inventors: Mark D. DEMARIO, New York, NY (US); Thomas FRIESS, Penzberg (DE); Astrid Alexandra RUEFLI-BRASSE, Basel (CH)

(73) Assignee: Hoffmann-La Roche Inc., Little Falls, NJ (US)

(21) Appl. No.: 16/752,537

(22) Filed: Jan. 24, 2020

Related U.S. Application Data
(63) Continuation of application No. PCT/EP2018/070001, filed on Jul. 24, 2018.
(60) Provisional application No. 62/537,127, filed on Jul. 26, 2017.

(51) Int. Cl. A61K 39/395; A61P 35/00; A61K 31/496; A61K 31/551

(52) U.S. Cl. CPC A61K 39/3955; A61K 31/551; A61K 31/496; A61P 35/00

(57) ABSTRACT

The present invention is directed to the combination therapy of DLBCL with a BET inhibitor, a Bcl-2 inhibitor and an anti-CD20 antibody.

### CROSS-REFERENCE TO RELATED APPLICATION

This application is a continuation of International Patent Application No. PCT/EP2018/070001, having an international filing date of Jul. 24, 2018, which claims benefit to U.S. Patent Application No. 62/537,127 filed Jul. 26, 2017, the entire contents of each are incorporated herein by reference in their entirety.

### SEQUENCE LISTING

This application contains a Sequence Listing which has been submitted via EFS-Web and is hereby incorporated by reference in its entirety. Said ASCII copy, created on Jan. 22, 2020, is named P34351-US-1_Sequence_Listing.txt and is 21,669 bytes in size.

### FIELD OF INVENTION

The present invention is directed to the combination therapy of cancer, in particular of DLBCL, with a BET inhibitor, a Bcl-2 inhibitor and an anti-CD20 antibody.

B-cell lymphomas are much more common than T-cell lymphomas and account for approximately 85 percent of all Non-Hodgkin lymphomas (NHLs). Diffuse large B-cell lymphoma (DLBCL) is the most common form of NHL, accounting for about 30 percent of newly diagnosed cases of NHL in the United States. DLBCL occurs in both men and women, although it is slightly more common in men. Although DLBCL can occur in childhood, its incidence generally increases with age, and roughly half of patients are over the age of 60.

DLBCL is an aggressive (fast-growing) lymphoma that can arise in lymph nodes or outside of the lymphatic system, in the gastrointestinal tract, testes, thyroid, skin, breast, bone, or brain. Often, the first sign of DLBCL is a painless, rapid swelling in the neck, underarms, or groin that is caused by enlarged lymph nodes. For some patients, the swelling may be painful. Other symptoms may include night sweats, fever, and unexplained weight loss. Patients may notice fatigue, loss of appetite, shortness of breath, or pain.

Epigenetic dysregulation plays an important role in driving the aberrant gene expression patterns seen in a variety of hematologic malignancies. As many epigenetic alterations are reversible, these factors have drawn considerable attention as potential antineoplastic targets. One particular target of significant clinical interest is the bromodomain and extra-terminal (BET) family of proteins, which includes BRD2, BRD3, BRD4, and the testis-specific BRDT. Bromodomains (BRDs) are protein domains that possess a high affinity for binding to acetylation motifs, including acetylated histone proteins within chromatin. The BET family of proteins binds to acetylated chromatin and regulates gene transcription.

Selective inhibition of the interaction between BET proteins and acetylated chromatin has resulted in significant activity in preclinical models of acute leukemia, lymphoma, and multiple myeloma (MM). Targeting BET proteins could specifically target transcription of oncogenes and genes critical to disease development and progression. (Onco Targets Ther. 2016; 9)

Bcl-2 proteins play a role in many diseases, particularly in cancer, leukemia, immune and autoimmune diseases. Bcl-2 proteins are said to be involved in bladder cancer, brain cancer, breast cancer, bone marrow cancer, cervical cancer, chronic lymphocytic leukemia, colorectal cancer, esophageal cancer, hepatocellular cancer, lymphoblastic leukemia, follicular lymphoma, lymphoid malignancies of T-cell or B-cell origin, melanoma, myelogenous leukemia, myeloma, oral cancer, ovarian cancer, non-small cell lung cancer, prostate cancer, small cell lung cancer, spleen cancer. Overexpression of Bcl-2 proteins correlate with resistance to chemotherapy, clinical outcome, disease progression, overall prognosis or a combination thereof in various cancers and disorders of the immune system.

The CD20 molecule (also called human B-lymphocyte-restricted differentiation antigen or Bp35) is a hydrophobic transmembrane protein located on pre-B and mature B lymphocytes that has been described extensively (Valentine, M. A., et al., J. Biol. Chem. 264 (1989) 11282-11287; and Einfeld, D. A., et al., EMBO J. 7 (1988) 711-717; Tedder, T. F., et al., Proc. Natl. Acad. Sci. U.S.A. 85 (1988) 208-212; Stamenkovic, I., et al., J. Exp. Med. 167 (1988) 1975-1980; Tedder, T. F., et al., J. Immunol. 142 (1989) 2560-2568). CD20 is expressed on greater than 90% of B cell non-Hodgkin's lymphomas (NHL) (Anderson, K. C., et al., Blood 63 (1984) 1424-1433) but is not found on hematopoietic stem cells, pro-B cells, normal plasma cells, or other normal tissues (Tedder, T. F., et al., J, Immunol. 135 (1985) 973-979).

There exist two different types of anti-CD20 antibodies differing significantly in their mode of CD20 binding and biological activities (Cragg, M. S., et al., Blood 103 (2004) 2738-2743; and Cragg, M. S., et al., Blood 101 (2003) 1045-1052). Type I anti-CD20 antibodies primarily utilize complement to kill target cells, while Type II antibodies primarily operate through direct induction of cell death.

Type I and Type II anti-CD20 antibodies and their characteristics are reviewed e.g. in Klein et al., mAbs 5 (2013), 22-33. Type II anti-CD20 antibodies do not localize CD20 to lipid rafts, show low CDC (complement dependent lysis) activity, show only about half the binding capacity to B cells as compared to Type I anti-CD20 antibodies, and induce homotypic aggregation and direct cell death. In contrast thereto, Type I antibodies localize CD20 to lipid rafts, show high CDC activity, full binding capacity to B cells, and only weak induction of homotypic aggregation and direct cell death.

Cell-mediated effector functions of monoclonal antibodies can be enhanced by engineering their oligosaccharide component as described in Umaña, P., et al., Nature Biotechnol. 17 (1999) 176-180; and U.S. Pat. No. 6,602,684. IgG1 type antibodies, the most commonly used antibodies in cancer immunotherapy, are glycoproteins that have a conserved N-linked glycosylation site at Asn297 in each CH2 domain. The two complex biantennary oligosaccharides attached to Asn297 are buried between the CH2 domains, forming extensive contacts with the polypeptide backbone, and their presence is essential for the antibody to mediate effector functions such as antibody dependent cellular cytotoxicity (ADCC) (Lifely, M. R., et al., Glycobiology 5 (1995) 813-822; Jefferis, R., et al., Immunol. Rev. 163 (1998) 59-76; Wright, A., and Morrison, S. L., Trends Biotechnol. 15 (1997) 26-32). Umaña, P., et al., Nature Biotechnol. 17 (1999) 176-180 and WO 99/154342 showed that overexpression in Chinese hamster ovary (CHO) cells of β(1,4)-N-acetylglucosaminyltransferase III (“GnTIII”), a glycosyltransferase catalyzing the formation of bisected oligosaccharides, significantly increases the in vitro ADCC activity of antibodies. Alterations in the composition of the N297 carbohydrate or its elimination affect also binding to Fc binding to FcγR and C1 q (Umaña, P., et al., Nature Biotechnol. 17 (1999) 176-180; Davies, J., et al., Biotechnol. Bioeng. 74 (2001) 288-294; Mimura, Y., et al., J. Biol. Chem. 276 (2001) 45539-45547; Radaev, S., et al., J. Biol. Chem. 276 (2001) 16478-16483; Shields, R. L., et al., J. Biol. Chem. 276 (2001) 6591-6604; Shields, R. L., et al., J. Biol. Chem. 277 (2002) 26733-26740; Simmons, L. C., et al., J. Immunol. Methods 263 (2002) 133-147).

Studies discussing the activities of afucosylated and fucosylated antibodies, including anti-CD20 antibodies, have been reported (e.g., lida, S., et al., Clin. Cancer Res. 12 (2006) 2879-2887; Natsume, A., et al., J. Immunol. Methods 306 (2005) 93-103; Satoh, M., et al., Expert Opin. Biol. Ther. 6 (2006) 1161-1173; Kanda, Y., et al., Biotechnol. Bioeng. 94 (2006) 680-688; Davies, J., et al., Biotechnol. Bioeng. 74 (2001) 288-294).

It was surprisingly found that the combination of a BET inhibitor with a Bcl-2 inhibitor and an anti-CD20 antibody showed significantly enhanced efficacy against DLBCL, causing a distinct tumor regression and a delay of tumor regrowth after stop of treatment. Surprisingly, the tumor regression with this triple combination is more than additive, i.e. superior to the cumulated tumor regression induced by each of the three components separately.

The invention thus relates in particular to:

A BET inhibitor, a Bcl-2 inhibitor and an anti-CD20 antibody for use as a medicament;

A BET inhibitor, a Bcl-2 inhibitor and an anti-CD20 antibody for use in the treatment of DLBCL;

The BET inhibitor, Bcl-2 inhibitor and anti-CD20 antibody for use according to the invention, wherein the BET inhibitor is 2-[(S)-4-(4-Chloro-phenyl)-2,3,9-trimethyl-6H-1-thia-5,7,8,9a-tetraaza-cyclopenta[e]azulen-6-yl]-N-[3-(4-methyl-piperazin-1-yl)-propyl]-acetamide (RG6146), INCB-054329, INCB-057643, GSK525762, GS-5829, CPI-0610, Birabresib, PLX51107, ABBV-075, BI 894999, FT-1101, ZEN-3694, GSK-2820151 or BMS-986158;

The BET inhibitor, Bcl-2 inhibitor and anti-CD20 antibody for use according to the invention, wherein the BET inhibitor is 2-[(S)-4-(4-Chloro-phenyl)-2,3,9-trimethyl-6H-1-thia-5,7,8,9a-tetraaza-cyclopenta[e]azulen-6-yl]-N-[3-(4-methyl-piperazin-1-yl)-propyl]-acetamide (RG6146);

The BET inhibitor, Bcl-2 inhibitor and anti-CD20 antibody for use according to the invention, wherein the Bcl-2 inhibitor is venetoclax, navitoclax, obatoclax, S-055746 or PNT-2258;

The BET inhibitor, Bcl-2 inhibitor and anti-CD20 antibody for use according to the invention, wherein the Bcl-2 inhibitor is venetoclax;

The BET inhibitor, Bcl-2 inhibitor and anti-CD20 antibody for use according to the invention, wherein the anti-CD20 antibody is a Type I anti-CD20 antibody, or a Type II anti-CD20 antibody wherein at least 40% of the N-linked oligosaccharides in the Fc region are non-fucosylated;

The BET inhibitor, Bcl-2 inhibitor and anti-CD20 antibody for use according to the invention, wherein the Type II anti-CD20 antibody is a humanized B-Ly1 antibody;

The BET inhibitor, Bcl-2 inhibitor and anti-CD20 antibody for use according to the invention, wherein the Type II anti-CD20 antibody is obinutuzumab;

The BET inhibitor, Bcl-2 inhibitor and anti-CD20 antibody for use according to the invention, wherein the type I anti-CD20 antibody is rituximab;

The BET inhibitor, Bcl-2 inhibitor and anti-CD20 antibody for use according to the invention, wherein the anti-CD20 antibody is rituximab or obinutzumab;

The BET inhibitor, Bcl-2 inhibitor and anti-CD20 antibody for use according to the invention, wherein the anti-CD20 antibody is rituximab;

The BET inhibitor, Bcl-2 inhibitor and anti-CD20 antibody for use according to the invention, wherein the anti-CD20 antibody is obinutzumab;

The BET inhibitor, Bcl-2 inhibitor and anti-CD20 antibody for use according to the invention, comprising one or more additional other cytotoxic, chemotherapeutic or anti-cancer agents;

The BET inhibitor, Bcl-2 inhibitor and anti-CD20 antibody for use according to the invention, comprising ionizing radiation enhancing the effects of said agents;

A pharmaceutical composition comprising a BET inhibitor, a Bcl-2 inhibitor and an anti-CD20 antibody and one or more pharmaceutically acceptable excipients;

A pharmaceutical composition comprising a BET inhibitor, a Bcl-2 inhibitor and an anti-CD20 antibody and one or more pharmaceutically acceptable excipients for use in the treatment of DLBCL;

The use of a BET inhibitor, a Bcl-2 inhibitor and an anti-CD20 antibody for the manufacture of a medicament for the treatment of DLBCL;

The use of a BET inhibitor, a Bcl-2 inhibitor and an anti-CD20 antibody in the treatment of DLBCL;

A method of treatment of DLBCL comprising the administering of a BET inhibitor, a Bcl-2 inhibitor and an anti-CD20 antibody to a patient in the need thereof;

A kit comprising a BET inhibitor, a Bcl-2 inhibitor and an anti-CD20 antibody for the simultaneous, separate or sequential administration of said BET inhibitor, Bcl-2 inhibitor and anti-CD20 antibody;

A kit according to the invention for use in the treatment of DLBCL;

A pharmaceutical composition, a use, a method or a kit according to the invention, wherein the BET inhibitor is 2-[(S)-4-(4-chloro-phenyl)-2,3,9-trimethyl-6H-1-thia-5,7,8,9a-tetraaza-cyclopenta[e] azulen-6-yl]-N-[3-(4-methyl-piperazin-1-yl)-propyl]-acetamide (RG6146), INCB-054329, INCB-057643, GSK525762, GS-5829, CPI-0610, Birabresib, PLX51107, ABBV-075, BI 894999, FT-1101, ZEN-3694, GSK-2820151 or BMS-986158;

A pharmaceutical composition, a use, a method or a kit according to the invention, wherein the BET inhibitor is RG6146;

A pharmaceutical composition, a use, a method or a kit according to the invention, wherein the Bcl-2 inhibitor is venetoclax, navitoclax, obatoclax, S-055746 or PNT-2258;

A pharmaceutical composition, a use, a method or a kit according to the invention, wherein the Bcl-2 inhibitor is venetoclax;

A pharmaceutical composition, a use, a method or a kit according to the invention, wherein the anti-CD20 antibody is a Type I anti-CD20 antibody, or a Type II anti-CD20 antibody wherein at least 40% of the N-linked oligosaccharides in the Fc region are non-fucosylated;

A pharmaceutical composition, a use, a method or a kit according to the invention, wherein the Type II anti-CD20 antibody is a humanized B-Ly1 antibody;

A pharmaceutical composition, a use, a method or a kit according to the invention, wherein the Type II anti-CD20 antibody is obinutuzumab;

A pharmaceutical composition, a use, a method or a kit according to the invention, wherein the Type I anti-CD20 antibody is rituximab;

A pharmaceutical composition, a use, a method or a kit according to the invention, wherein the anti-CD20 antibody is rituximab or obinutuzumab;

A pharmaceutical composition, a use, a method or a kit according to the invention, wherein the anti-CD20 antibody is rituximab; and

A pharmaceutical composition, a use, a method or a kit according to the invention, wherein the anti-CD20 antibody is obinutuzumab.

The BET inhibitor, Bcl-2 inhibitor and anti-CD20 antibody for use according to the invention are thus administered in combination (or co-administered).

The invention thus relates to a BET inhibitor, a Bcl-2 inhibitor and an anti-CD20 antibody for use in combination according to the invention.

The invention thus relates to a BET inhibitor, a Bcl-2 inhibitor and an anti-CD20 antibody for use in combination as a medicament, in particular for use in combination in the treatment of DLBCL.

In one embodiment, the BET inhibitor is a compound selected from the compounds described in WO 2011/143669. Methods of producing said BET inhibitors are also disclosed in WO 2011/143669.

Most preferably, the BET inhibitor is 2-[(S)-4-(4-Chloro-phenyl)-2,3,9-trimethyl-6H-1-thia-5,7,8,9a-tetraaza-cyclopenta[e]azulen-6-yl]-N-[3-(4-methyl-piperazin-1-yl)-propyl]-acetamide as in the formula below or a salt thereof. Example JQ35 of WO 2011/143669 describes a method for its preparation.

The preferred BET inhibitor is depicted in the following formula:

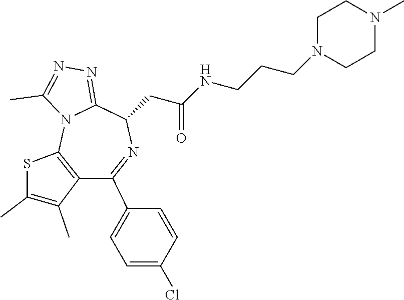

The above BET inhibitor is also known as RG6146, JQ35 or TEN-010.

In one embodiment, the Bcl-2 inhibitor is a compound selected from the compounds described in WO 2010/138588. Methods of producing said Bcl-2 inhibitors are also disclosed in WO 2010/138588.

Most preferably, the Bcl-2 inhibitor is 4-(4-{[2-(4-chlorophenyl)-4,4-dimethylcyclohex-1-en-1-yl]methyl}piperazin-1-yl)-N-({3-nitro-4-[(tetrahydro-2H-pyran-4-ylmethyl)amino]phenyl}sulfonyl)-2-(1H-pyrrolo[2,3-b]pyridin-5-yloxy)benzamide as in the formula below or a salt thereof. Example 5 of WO 2010/138588 describes methods for preparation of said Bcl-2 inhibitor.

The preferred Bcl-2 inhibitor is depicted in the following formula:

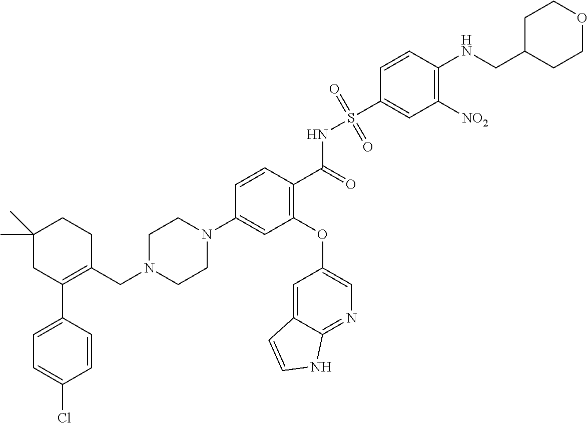 [SMILES: CC1(C)CCC(CN2CCN(c3ccc(C(=O)NS(=O)(=O)c4ccc(NCC5CCOCC5)c([N+](=O)[O-])c4)c(Oc4cnc5[nH]ccc5c4)c3)CC2)=C(c2ccc(Cl)cc2)C1]

The above Bcl-2 inhibitor is also named ABT-199, GDC-0199 or venetoclax.

The anti-CD20 antibody can be a Type I anti-CD20 antibody or a Type II anti-CD20 antibody.

Rituximab is a particularly preferred anti-CD20 antibody. It is a Type I anti-CD20 antibody. It is a genetically engineered chimeric human gamma 1 murine constant domain containing monoclonal antibody directed against the human CD20 antigen. This chimeric antibody contains human gamma 1 constant domains and is identified by the name “C2B8” in U.S. Pat. No. 5,736,137 (Anderson et. al.) issued on Apr. 7, 1998, assigned to IDEC Pharmaceuticals Corporation. Rituximab is approved for the treatment of patients with relapsed or refracting low-grade or follicular, CD20 positive, B cell non-Hodgkin's lymphoma. In vitro mechanism of action studies have shown that rituximab exhibits human complement-dependent cytotoxicity (CDC) (Reff, M. E., et. al., Blood 83 (1994) 435-445). Additionally, it exhibits significant activity in assays that measure antibody-dependent cellular cytotoxicity (ADCC). Rituximab is not afucosylated.

The Type II anti-CD20 antibody is advantageously engineered by modification of the glycosylation in the Fc region. In a specific embodiment the Type II anti-CD20 antibody is engineered to have an increased proportion of non-fucosylated oligosaccharides in the Fc region as compared to a non-engineered antibody. An increased proportion of non-fucosylated oligosaccharides in the Fc region of an antibody results in the antibody having increased effector function.

In a more specific embodiment, at least about 40%, about 45%, about 50%, about 55%, about 60%, about 65%, about 70%, about 75%, about 80%, about 85%, about 90%, about 95% or about 100%, preferably at least about 40%, of the N-linked oligosaccharides in the Fc region of the Type II anti-CD20 antibody are non-fucosylated

In one embodiment, between about 40% and about 80% of the N-linked oligosaccharides in the Fc region of the Type II anti-CD20 antibody are non-fucosylated. In one embodiment, between about 40% and about 60% of the N-linked oligosaccharides in the Fc region of the Type II anti-CD20 antibody are non-fucosylated.

In another specific embodiment the Type II anti-CD20 antibody is engineered to have an increased proportion of bisected oligosaccharides in the Fc region as compared to a non-engineered antibody. In a more specific embodiment, at least about 10%, about 15%, about 20%, about 25%, about 30%, about 35%, about 40%, about 45%, about 50%, about 55%, about 60%, about 65%, about 70%, about 75%, about 80%, about 85%, about 90%, about 95% or about 100%, preferably at least about 40%, of the N-linked oligosaccharides in the Fc region of the Type II anti-CD20 antibody are bisected. In one embodiment, between about 40% and about 80% of the N-linked oligosaccharides in the Fc region of the anti-CD20 antibody are bisected. In one embodiment, between about 40% and about 60% of the N-linked oligosaccharides in the Fc region of the Type II anti-CD20 antibody are bisected.

The anti-CD20 antibody is advantageously a humanized B-Ly1 antibody.

In one embodiment, the humanized B-Ly1 antibody has a variable region of the heavy chain (VH) selected from group of of SEQ ID NO:3 to SEQ ID NO: 19 (B-HH2 to B-HH9 and B-HL8 to B-HL17 of WO 2005/044859 and WO 2007/031875).

In one specific embodiment, such variable domain is selected from the group consisting of SEQ ID NO:3, SEQ ID NO:4, SEQ ID NO:7, SEQ ID NO:9, SEQ ID NO: 11, SEQ ID NO:13 and SEQ ID NO:15 (B-HH2, BHH-3, B-HH6, B-HH8, B-HL8, B-HL11 and B-HL13 of WO 2005/044859 and WO 2007/031875).

In one specific embodiment, the humanized B-Ly1 antibody has a variable region of the heavy chain (VH) of SEQ ID NO:7 (B-HH6 of WO 2005/044859 and WO 2007/031875).

In one specific embodiment, the humanized B-Ly1 antibody has a variable region of the light chain (VL) of SEQ ID NO:20 (B-KV1 of WO 2005/044859 and WO 2007/031875).

In one specific embodiment, the humanized B-Ly1 antibody has a variable region of the heavy chain (VH) of SEQ ID NO:7 (B-HH6 of WO 2005/044859 and WO 2007/031875) and a variable region of the light chain (VL) of SEQ ID NO:20 (B-KV1 of WO 2005/044859 and WO 2007/031875).

Furthermore, in one embodiment, the humanized B-Ly1 antibody is an IgG1 antibody.

According to the invention such humanized B-Ly1 antibodies are preferrably glycoengineered (GE) in the Fc region according to the procedures described in WO 2005/044859, WO 2004/065540, WO 2007/031875, Umana, P. et al., Nature Biotechnol. 17 (1999) 176-180 and WO 99/154342.

In one embodiment, the glyco-engineered humanized B-Ly1 is B-HH6-B-KV1 GE.

In one embodiment, the anti-CD20 antibody is obinutuzumab (recommended INN, WHO Drug Information, Vol. 26, No. 4, 2012, p. 453). As used herein, obinutuzumab is synonymous for GA101 and is formerly known as afutuzumab (recommended INN, WHO Drug Information, Vol. 23, No. 2, 2009, p. 176; Vol. 22, No. 2, 2008, p. 124). The tradename is GAZYVA or GAZYVARO. The WHO Drug Information document replaces all previous versions (e.g. Vol. 25, No. 1, 2011, p. 75-76).

In one embodiment, the Type II anti-CD20 antibody binds CD20 with a KD of 10⁻⁸ M to 10⁻¹³ M.

In a particular aspect of the invention, the Type II anti-CD20 antibody is of IgG1 isotype.

In a particular aspect of the invention, the Type II anti-CD20 antibody is a humanized B-Ly1 antibody.

In a particularly preferred embodiment, the Type II anti-CD20 antibody is obinutuzumab.

### BRIEF DESCRIPTION OF THE DRAWINGS

FIG. 1: Antitumor efficacy of therapy with the triple combination of RG6146, venetoclax and obinutzumab, compared to vehicule, mono and dual therapy (Day 10-50).

FIG. 2: Tumor growth delay after treatment with the triple combination of RG6146, venetoclax and obinutuzumab, compared the the vehicule, mono and dual therapy.

FIG. 3: Antitumor efficacy of therapy with the triple combination of RG6146, venetoclax and rituximab, compared to vehicule, mono and dual therapy (Day 10-28).

The term “antibody” encompasses the various forms of antibodies including but not being limited to whole antibodies, human antibodies, humanized antibodies and genetically engineered antibodies like monoclonal antibodies, chimeric antibodies or recombinant antibodies as well as fragments of such antibodies as long as the characteristic properties according to the invention are retained. The terms “monoclonal antibody” or “monoclonal antibody composition” as used herein refer to a preparation of antibody molecules of a single amino acid composition. Accordingly, the term “human monoclonal antibody” refers to antibodies displaying a single binding specificity which have variable and constant regions derived from human germline immunoglobulin sequences. In one embodiment, the human monoclonal antibodies are produced by a hybridoma which includes a B cell obtained from a transgenic non-human animal, e.g. a transgenic mouse, having a genome comprising a human heavy chain transgene and a light human chain transgene fused to an immortalized cell.

The term “chimeric antibody” refers to a monoclonal antibody comprising a variable region, i.e., binding region, from one source or species and at least a portion of a constant region derived from a different source or species, usually prepared by recombinant DNA techniques. Chimeric antibodies comprising a murine variable region and a human constant region are especially preferred. Such murine/human chimeric antibodies are the product of expressed immunoglobulin genes comprising DNA segments encoding murine immunoglobulin variable regions and DNA segments encoding human immunoglobulin constant regions. Other forms of “chimeric antibodies” encompassed by the present invention are those in which the class or subclass has been modified or changed from that of the original antibody. Such “chimeric” antibodies are also referred to as “class-switched antibodies.” Methods for producing chimeric antibodies involve conventional recombinant DNA and gene transfection techniques now well known in the art. See, e.g., Morrison, S. L., et al., Proc. Natl. Acad Sci. USA 81 (1984) 6851-6855; U.S. Pat. Nos. 5,202,238 and 5,204,244.

The term “humanized antibody” refers to antibodies in which the framework or “complementarity determining regions” (CDR) have been modified to comprise the CDR of an immunoglobulin of different specificity as compared to that of the parent immunoglobulin. In a preferred embodiment, a murine CDR is grafted into the framework region of a human antibody to prepare the “humanized antibody.” See, e.g., Riechmann, L. et al., Nature 332 (1988) 323-327; and Neuberger, M. S. et al., Nature 314 (1985) 268-270. Particularly preferred CDRs correspond to those representing sequences recognizing the antigens noted above for chimeric and bi- or multispecific antibodies.

The term “human antibody”, as used herein, is intended to include antibodies having variable and constant regions derived from human germline immunoglobulin sequences. Human antibodies are well-known in the state of the art (van Dijk, M. A., and van de Winkel, J. G., Curr. Opin. in Chem. Biol. 5 (2001) 368-374). Based on such technology, human antibodies against a great variety of targets can be produced. Examples of human antibodies are for example described in Kellermann, S. A., et al., Curr Opin Biotechnol. 13 (2002) 593-597.

The term “recombinant human antibody”, as used herein, is intended to include all human antibodies that are prepared, expressed, created or isolated by recombinant means, such as antibodies isolated from a host cell such as a NSO or CHO cell or from an animal (e.g. a mouse) that is transgenic for human immunoglobulin genes or antibodies expressed using a recombinant expression vector transfected into a host cell. Such recombinant human antibodies have variable and constant regions derived from human germline immunoglobulin sequences in a rearranged form. The recombinant human antibodies according to the invention have been subjected to in vivo somatic hypermutation. Thus, the amino acid sequences of the VH and VL regions of the recombinant antibodies are sequences that, while derived from and related to human germline VH and VL sequences, may not naturally exist within the human antibody germline repertoire in vivo.

The term “bi- or multispecific antibody” as used herein relates to monoclonal antibodies that have binding specificities for at least two different sites. In certain embodiments, one of the binding specificities is for CD20 and the other is for any other antigen. In certain embodiments, bispecific antibodies may bind to two different epitopes of CD20. Bispecific antibodies may also be used to localize cytotoxic agents to cells which express CD20. Bispecific antibodies can be prepared as full length antibodies or antibody fragments.

The terms “full length antibody,” “intact antibody,” and “whole antibody” are used herein interchangeably to refer to an antibody having a structure substantially similar to a native antibody structure or having heavy chains that contain an Fc region as defined herein.

An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)2, diabodies, linear antibodies, single-chain antibody molecules (e.g. scFv), and multispecific antibodies formed from antibody fragments. The term “antibody fragment” as used herein also encompasses single-domain antibodies.

The term “Fc domain” or “Fc region” herein is used to define a C-terminal region of an immunoglobulin heavy chain that contains at least a portion of the constant region. The term includes native sequence Fc regions and variant Fc regions. Although the boundaries of the Fc region of an IgG heavy chain might vary slightly, the human IgG heavy chain Fc region is usually defined to extend from Cys226, or from Pro230, to the carboxyl-terminus of the heavy chain. However, antibodies produced by host cells may undergo post-translational cleavage of one or more, particularly one or two, amino acids from the C-terminus of the heavy chain. Therefore, an antibody produced by a host cell by expression of a specific nucleic acid molecule encoding a full-length heavy chain may include the full-length heavy chain, or it may include a cleaved variant of the full-length heavy chain (also referred to herein as a “cleaved variant heavy chain”). This may be the case where the final two C-terminal amino acids of the heavy chain are glycine (G446) and lysine (K447, numbering according to Kabat EU index). Therefore, the C-terminal lysine (Lys447), or the C-terminal glycine (Gly446) and lysine (K447), of the Fc region may or may not be present. Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, Md., 1991 (see also above). A “subunit” of an Fc domain as used herein refers to one of the two polypeptides forming the dimeric Fc domain, i.e. a polypeptide comprising C-terminal constant regions of an immunoglobulin heavy chain, capable of stable self-association. For example, a subunit of an IgG Fc domain comprises an IgG CH2 and an IgG CH3 constant domain.

As used herein, the term “binding” or “specifically binding”, when characterizing an antibody, refers to the binding of the antibody to an epitope of the tumor antigen in an in vitro assay, preferably in an plasmon resonance assay (BIAcore, GE-Healthcare Uppsala, Sweden) with purified wild-type antigen. The affinity of the binding is defined by the terms ka (rate constant for the association of the antibody from the antibody/antigen complex), k_{D} (dissociation constant), and K_{D} (k_{D}/ka). Binding or specifically binding means a binding affinity (K_{D}) of 10⁻⁸ M or less, preferably 10⁻⁸ M to 10⁻¹³ M (in one embodiment 10⁻⁹ M to 10⁻¹³ M). Thus, an afucosylated antibody according to the invention is specifically binding to the tumor antigen with a binding affinity (K_{D}) of 10⁻⁸ mol/l or less, preferably 10⁻⁸ M to 10⁻¹³ M (in one embodiment 10⁻⁹ M to 10⁻¹³ M).

The term “nucleic acid molecule”, as used herein, is intended to include DNA molecules and RNA molecules. A nucleic acid molecule may be single-stranded or double-stranded, but preferably is double-stranded DNA.

The “constant domains” are not involved directly in binding the antibody to an antigen but are involved in the effector functions (ADCC, complement binding, and CDC).

The “variable region” (variable region of a light chain (VL), variable region of a heavy chain (VH)) as used herein denotes each of the pair of light and heavy chains which is involved directly in binding the antibody to the antigen. The domains of variable human light and heavy chains have the same general structure and each domain comprises four framework (FR) regions whose sequences are widely conserved, connected by three “hypervariable regions” (or complementarity determining regions, CDRs). The framework regions adopt a β-sheet conformation and the CDRs may form loops connecting the β-sheet structure. The CDRs in each chain are held in their three-dimensional structure by the framework regions and form together with the CDRs from the other chain the antigen binding site.

The terms “hypervariable region” when used herein refer to the amino acid residues of an antibody which are responsible for antigen-binding. The hypervariable region comprises amino acid residues from the “complementarity determining regions” or “CDRs”. “Framework” or “FR” regions are those variable domain regions other than the hypervariable region residues as herein defined. Therefore, the light and heavy chains of an antibody comprise from N- to C-terminus the domains FR1, CDR1, FR2, CDR2, FR3, CDR3, and FR4. Especially, CDR3 of the heavy chain is the region which contributes most to antigen binding. CDR and FR regions are determined according to the standard definition of Kabat, et al., Sequences of Proteins of Immunological Interest, 5th ed., Public Health Service, National Institutes of Health, Bethesda, Md. (1991), and/or those residues from a “hypervariable loop”.

The term “afucosylated antibody” refers to an antibody of IgG1 or IgG3 isotype (preferably of IgG1 isotype) with an altered pattern of glycosylation in the Fc region at Asn297 having a reduced level of fucose residues. Glycosylation of human IgG1 or IgG3 occurs at Asn297 as core fucosylated bianntennary complex oligosaccharide glycosylation terminated with up to 2 Gal residues. These structures are designated as G0, G1 (α1,6 or α1,3) or G2 glycan residues, depending from the amount of terminal Gal residues (Raju, T. S., BioProcess Int. 1 (2003) 44-53). CHO type glycosylation of antibody Fc parts is e.g. described by Routier, F. H., Glycoconjugate J. 14 (1997) 201-207. Antibodies which are recombinantely expressed in non glycomodified CHO host cells usually are fucosylated at Asn297 in an amount of at least 85%. It should be understood that the term an afucosylated antibody as used herein includes an antibody having no fucose in its glycosylation pattern. It is commonly known that typical glycosylated residue position in an antibody is the asparagine at position 297 according to the EU numbering system (“Asn297”).

The “EU numbering system” or “EU index” is generally used when referring to a residue in an immunoglobulin heavy chain constant region (e.g., the EU index reported in Kabat et al., Sequences of Proteins of Immunological Interest, 5th ed., Public Health Service, National Institutes of Health, Bethesda, Md. (1991) expressly incorporated herein by reference).

CD20 (also known as B-lymphocyte antigen CD20, B-lymphocyte surface antigen B1, Leu-16, Bp35, BM5, and LF5; the sequence is characterized by the SwissProt database entry P11836) is a hydrophobic transmembrane protein with a molecular weight of approximately 35 kD located on pre-B and mature B lymphocytes (Valentine, M. A. et al., J. Biol. Chem. 264 (1989) 11282-11287; Tedder, T. F., et al., Proc. Natl. Acad. Sci. U.S.A. 85 (1988) 208-212; Stamenkovic, I., et al., J. Exp. Med. 167 (1988) 1975-1980; Einfeld, D. A., et al., EMBO J. 7 (1988) 711-717; Tedder, T. F., et al., J. Immunol. 142 (1989) 2560-2568). The corresponding human gene is Membrane-spanning 4-domains, subfamily A, member 1, also known as MS4A1. This gene encodes a member of the membrane-spanning 4A gene family. Members of this nascent protein family are characterized by common structural features and similar intron/exon splice boundaries and display unique expression patterns among hematopoietic cells and nonlymphoid tissues. This gene encodes the B-lymphocyte surface molecule which plays a role in the development and differentiation of B-cells into plasma cells. This family member is localized to 11q12, among a cluster of family members. Alternative splicing of this gene results in two transcript variants which encode the same protein.

The terms “CD20” and “CD20 antigen” are used interchangeably herein, and include any variants, isoforms and species homologs of human CD20 which are naturally expressed by cells or are expressed on cells transfected with the CD20 gene. Binding of an antibody of the invention to the CD20 antigen mediate the killing of cells expressing CD20 (e.g., a tumor cell) by inactivating CD20. The killing of the cells expressing CD20 may occur by one or more of the following mechanisms: Cell death/apoptosis induction, ADCC and CDC.

Synonyms of CD20, as recognized in the art, include B-lymphocyte antigen CD20, B-lymphocyte surface antigen B1, Leu-16, Bp35, BM5, and LF5.

The term “anti-CD20 antibody” according to the invention is an antibody that binds specifically to CD20 antigen. Depending on binding properties and biological activities of anti-CD20 antibodies to the CD20 antigen, two types of anti-CD20 antibodies (type I and type II anti-CD20 antibodies) can be distinguished according to Cragg, M. S., et al., Blood 103 (2004) 2738-2743; and Cragg, M. S., et al., Blood 101 (2003) 1045-1052, see Table 1.

TABLE 1
Properties of Type I and Type II anti-CD20 antibodies

| Type I anti-CD20 antibodies | Type II anti-CD20 antibodies |
|---|---|
| type I CD20 epitope | type II CD20 epitope |
| Localize CD20 to lipid rafts | Do not localize CD20 to lipid rafts |
| Increased CDC (if IgG1 isotype) | Decreased CDC (if IgG1 isotype) |
| ADCC activity (if IgG1 isotype) | ADCC activity (if IgG1 isotype) |
| Full binding capacity | Reduced binding capacity |
| Homotypic aggregation | Stronger homotypic aggregation |
| Apoptosis induction upon cross- | Strong cell death induction without |
| linking | cross-linking |

Examples of Type I anti-CD20 antibodies include e.g. rituximab (a non-afucosylated antibody with an amount of fucose of 85% or higher), HI47 IgG3 (ECACC, hybridoma), 2C6 IgG1 (as disclosed in WO 2005/103081), 2F2 IgG1 (as disclosed and WO 2004/035607 and WO 2005/103081), 2H7 IgG1 (as disclosed in WO 2004/056312), ofatumumab, veltuzumab, ocrelizumab, ocaratuzumab, PRO 131921 and ublituximab.

Examples of Type II anti-CD20 antibodies include e.g. humanized B-Ly1 antibodies, humanized B-Ly1 antibody IgG1 (a chimeric humanized IgG1 antibody as disclosed in WO 2005/044859), obinutuzumab, tositumumab (B1), 11B8 IgG1 (as disclosed in WO 2004/035607), AT80 IgG1. Typically, Type II anti-CD20 antibodies of the IgG1 isotype show characteristic CDC properties. Type II anti-CD20 antibodies have a decreased CDC (if IgG1 isotype) compared to type I antibodies of the IgG1 isotype.

The term “effector functions” when used in reference to antibodies refer to those biological activities attributable to the Fc region of an antibody, which vary with the antibody isotype. Examples of antibody effector functions include: C1q binding and complement dependent cytotoxicity (CDC), Fc receptor binding, antibody-dependent cell-mediated cytotoxicity (ADCC), antibody-dependent cellular phagocytosis (ADCP), cytokine secretion, immune complex-mediated antigen uptake by antigen presenting cells, down regulation of cell surface receptors (e.g. B cell receptor), and B cell activation.

Increased effector function can be measured by methods known in the art. A suitable assay for measuring ADCC is described herein. Other examples of in vitro assays to assess ADCC activity of a molecule of interest are described in U.S. Pat. No. 5,500,362; Hellstrom et al. Proc Natl Acad Sci USA 83, 7059-7063 (1986) and Hellstrom et al., Proc Natl Acad Sci USA 82, 1499-1502 (1985); U.S. Pat. No. 5,821,337; Bruggemann et al., J Exp Med 166, 1351-1361 (1987). Alternatively, non-radioactive assays methods may be employed (see, for example, ACTI™ non-radioactive cytotoxicity assay for flow cytometry (Cell Technology, Inc. Mountain View, Calif.); and CytoTox 96® non-radioactive cytotoxicity assay (Promega, Madison, Wis.)). Useful effector cells for such assays include peripheral blood mononuclear cells (PBMC) and Natural Killer (NK) cells. Alternatively, or additionally, ADCC activity of the molecule of interest may be assessed in vivo, e.g. in a animal model such as that disclosed in Clynes et al., Proc Natl Acad Sci USA 95, 652-656 (1998). Binding to Fc receptors can be easily determined e.g. by ELISA, or by Surface Plasmon Resonance (SPR) using standard instrumentation such as a BIAcore instrument (GE Healthcare), and Fc receptors such as may be obtained by recombinant expression. According to a particular embodiment, binding affinity to an activating Fc receptor is measured by surface plasmon resonance using a BIACORE® T100 machine (GE Healthcare) at 25° C. Alternatively, binding affinity of antibodies for Fc receptors may be evaluated using cell lines known to express particular Fc receptors, such as NK cells expressing FcγIIIa receptor. C1q binding assays may also be carried out to determine whether the antibody is able to bind C1q and hence has CDC activity. See e.g., C1q and C3c binding ELISA in WO 2006/029879 and WO 2005/100402. To assess complement activation, a CDC assay may be performed (see, for example, Gazzano-Santoro et al., J Immunol Methods 202, 163 (1996); Cragg et al., Blood 101, 1045-1052 (2003); and Cragg and Glennie, Blood 103, 2738-2743 (2004)).

One accepted in vitro ADCC assay is as follows:
1) the assay uses target cells that are known to express the target antigen recognized by the antigen-binding region of the antibody;
2) the assay uses human peripheral blood mononuclear cells (PBMCs), isolated from blood of a randomly chosen healthy donor, as effector cells;
3) the assay is carried out according to following protocol:
   i) the PBMCs are isolated using standard density centrifugation procedures and are suspended at 5×10⁶ cells/ml in RPMI cell culture medium;
   ii) the target cells are grown by standard tissue culture methods, harvested from the exponential growth phase with a viability higher than 90%, washed in RPMI cell culture medium, labeled with 100 micro-Curies of ⁵¹Cr, washed twice with cell culture medium, and resuspended in cell culture medium at a density of 10⁵ cells/ml;
   iii) 100 microliters of the final target cell suspension above are transferred to each well of a 96-well microtiter plate;
   iv) the antibody is serially-diluted from 4000 ng/ml to 0.04 ng/ml in cell culture medium and 50 microliters of the resulting antibody solutions are added to the target cells in the 96-well microtiter plate, testing in triplicate various antibody concentrations covering the whole concentration range above;
   v) for the maximum release (MR) controls, 3 additional wells in the plate containing the labeled target cells, receive 50 microliters of a 2% (VN) aqueous solution of non-ionic detergent (Nonidet, Sigma, St. Louis), instead of the antibody solution (point iv above);
   vi) for the spontaneous release (SR) controls, 3 additional wells in the plate containing the labeled target cells, receive 50 microliters of RPMI cell culture medium instead of the antibody solution (point iv above);
   vii) the 96-well microtiter plate is then centrifuged at 50×g for 1 minute and incubated for 1 hour at 4° C.;
   viii) 50 microliters of the PBMC suspension (point i above) are added to each well to yield an effector:target cell ratio of 25:1 and the plates are placed in an incubator under 5% CO₂ atmosphere at 37° C. for 4 hours;
   ix) the cell-free supernatant from each well is harvested and the experimentally released radioactivity (ER) is quantified using a gamma counter;
   x) the percentage of specific lysis is calculated for each antibody concentration according to the formula (ER-MR)/(MR-SR)×100, where ER is the average radioactivity quantified (see point ix above) for that antibody concentration, MR is the average radioactivity quantified (see point ix above) for the MR controls (see point v above), and SR is the average radioactivity quantified (see point ix above) for the SR controls (see point vi above);
4) “increased ADCC” is defined as either an increase in the maximum percentage of specific lysis observed within the antibody concentration range tested above, and/or a reduction in the concentration of antibody required to achieve one half of the maximum percentage of specific lysis observed within the antibody concentration range tested above. The increase in ADCC is relative to the ADCC, measured with the above assay, mediated by the same antibody, produced by the same type of host cells, using the same standard production, purification, formulation and storage methods, which are known to those skilled in the art, but that has not been produced by host cells engineered to overexpress GnTIII.

The “increased ADCC” can be obtained by glycoengineering of said antibodies, that means enhance said natural, cell-mediated effector functions of monoclonal antibodies by engineering their oligosaccharide component as described in Umana, P., et al., Nature Biotechnol. 17 (1999) 176-180 and U.S. Pat. No. 6,602,684.

The term “complement-dependent cytotoxicity (CDC)” refers to lysis of human tumor target cells by the antibody according to the invention in the presence of complement. CDC is measured preferably by the treatment of a preparation of CD20 expressing cells with an anti-CD20 antibody according to the invention in the presence of complement. CDC is found if the antibody induces at a concentration of 100 nM the lysis (cell death) of 20% or more of the tumor cells after 4 hours. The assay is performed preferably with ⁵¹Cr or Eu labeled tumor cells and measurement of released ⁵¹Cr or Eu. Controls include the incubation of the tumor target cells with complement but without the antibody.

The term “humanized B-Ly1 antibody” refers to humanized B-Ly1 antibody as disclosed in WO 2005/044859 and WO 2007/031875, which were obtained from the murine monoclonal anti-CD20 antibody B-Ly1 (variable region of the murine heavy chain (VH): SEQ ID NO:1; variable region of the murine light chain (VL): SEQ ID NO:2 (see Poppema, S. and Visser, L., Biotest Bulletin 3 (1987) 131-139) by chimerization with a human constant domain from IgG1 and following humanization (see WO 2005/044859 and WO 2007/031875). These “humanized B-Ly1 antibodies” are disclosed in detail in WO 2005/044859 and WO 2007/031875.

The term “BET inhibitor” according to the invention refers to an agent that prevents activity of BET proteins with an IC₅₀ of about 0.001 μM to about 2 μM.

The term “Bcl-2 inhibitor” according to the invention refers to an agent that prevents activity of Bcl-2 proteins with an IC₅₀ of about 0.001 μM to about 2 μM

“Salt” refers to salts of the compounds as a pharmaceutically acceptable salt. Such salts can be exemplified by the salts with alkali metals (potassium, sodium, and the like), salts with alkaline-earth metals (calcium, magnesium, and the like), the ammonium salt, salts with pharmaceutically acceptable organic amines (tetramethylammonium, triethylamine, methylamine, dimethylamine, cyclopentylamine, benzylamine, phenethylamine, piperidine, monoethanolamine, diethanolamine, tris(hydroxymethyl)aminomethane, lysine, arginine, N-methyl-D-glucamine, and the like), and acid addition salts (inorganic acid salts (the hydrochloride, hydrobromide, hydroiodide, sulfate, phosphate, nitrate, and the like) and organic acid salts (the acetate, trifluoroacetate, lactate, tartrate, oxalate, fumarate, maleate, benzoate, citrate, methanesulfonate, ethanesulfonate, benzenesulfonate, toluenesulfonate, isethionate, glucuronate, gluconate, and the like)).

“IC₅₀” refers to the concentration of a particular compound required to inhibit 50% of a specific measured activity.

The oligosaccharide component can significantly affect properties relevant to the efficacy of a therapeutic glycoprotein, including physical stability, resistance to protease attack, interactions with the immune system, pharmacokinetics, and specific biological activity. Such properties may depend not only on the presence or absence, but also on the specific structures, of oligosaccharides. Some generalizations between oligosaccharide structure and glycoprotein function can be made. For example, certain oligosaccharide structures mediate rapid clearance of the glycoprotein from the bloodstream through interactions with specific carbohydrate binding proteins, while others can be bound by antibodies and trigger undesired immune reactions (Jenkins, N., et al., Nature Biotechnol. 14 (1996) 975-981).

Mammalian cells are the excellent hosts for production of therapeutic glycoproteins, due to their capability to glycosylate proteins in the most compatible form for human application (Cumming, D. A., et al., Glycobiology 1 (1991) 115-130; Jenkins, N., et al., Nature Biotechnol. 14 (1996) 975-981). Bacteria very rarely glycosylate proteins, and like other types of common hosts, such as yeasts, filamentous fungi, insect and plant cells, yield glycosylation patterns associated with rapid clearance from the blood stream, undesirable immune interactions, and in some specific cases, reduced biological activity. Among mammalian cells, Chinese hamster ovary (CHO) cells have been most commonly used during the last two decades. In addition to giving suitable glycosylation patterns, these cells allow consistent generation of genetically stable, highly productive clonal cell lines. They can be cultured to high densities in simple bioreactors using serum free media, and permit the development of safe and reproducible bioprocesses. Other commonly used animal cells include baby hamster kidney (BHK) cells, NSO- and SP2/0-mouse myeloma cells. More recently, production from transgenic animals has also been tested (Jenkins, N., et al., Nature Biotechnol. 14 (1996) 975-981).

All antibodies contain carbohydrate structures at conserved positions in the heavy chain constant regions, with each isotype possessing a distinct array of N-linked carbohydrate structures, which variably affect protein assembly, secretion or functional activity (Wright, A., and Morrison, S. L., Trends Biotech. 15 (1997) 26-32). The structure of the attached N-linked carbohydrate varies considerably, depending on the degree of processing, and can include high-mannose, multiply-branched as well as biantennary complex oligosaccharides (Wright, A., and Morrison, S. L., Trends Biotech. 15 (1997) 26-32). Typically, there is heterogeneous processing of the core oligosaccharide structures attached at a particular glycosylation site such that even monoclonal antibodies exist as multiple glycoforms. Likewise, it has been shown that major differences in antibody glycosylation occur between cell lines, and even minor differences are seen for a given cell line grown under different culture conditions (Lifely, M. R., et al., Glycobiology 5 (1995) 813-822).

One way to obtain large increases in potency, while maintaining a simple production process and potentially avoiding significant, undesirable side effects, is to enhance the natural, cell-mediated effector functions of monoclonal antibodies by engineering their oligosaccharide component as described in Umana, P. et al., Nature Biotechnol. 17 (1999) 176-180 and U.S. Pat. No. 6,602,684. IgG1 type antibodies, the most commonly used antibodies in cancer immunotherapy, are glycoproteins that have a conserved N-linked glycosylation site at Asn297 in each CH2 domain. The two complex biantennary oligosaccharides attached to Asn297 are buried between the CH2 domains, forming extensive contacts with the polypeptide backbone, and their presence is essential for the antibody to mediate effector functions such as antibody dependent cellular cytotoxicity (ADCC) (Lifely, M. R., et al., Glycobiology 5 (1995) 813-822; Jefferis, R., et al., Immunol. Rev. 163 (1998) 59-76; Wright, A. and Morrison, S. L., Trends Biotechnol. 15 (1997) 26-32).

It was previously shown that overexpression in Chinese hamster ovary (CHO) cells of β(1,4)-N-acetylglucosaminyltransferase III (“GnTIII7y), a glycosyltransferase catalyzing the formation of bisected oligosaccharides, significantly increases the in vitro ADCC activity of an antineuroblastoma chimeric monoclonal antibody (chCE7) produced by the engineered CHO cells (see Umana, P. et al., Nature Biotechnol. 17 (1999) 176-180; and WO 99/154342, the entire contents of which are hereby incorporated by reference). The antibody chCE7 belongs to a large class of unconjugated monoclonal antibodies which have high tumor affinity and specificity, but have too little potency to be clinically useful when produced in standard industrial cell lines lacking the GnTIII enzyme (Umana, P., et al., Nature Biotechnol. 17 (1999) 176-180). That study was the first to show that large increases of ADCC activity could be obtained by engineering the antibody producing cells to express GnTIII, which also led to an increase in the proportion of constant region (Fc)-associated, bisected oligosaccharides, including bisected, non-fucosylated oligosaccharides, above the levels found in naturally-occurring antibodies.

The term “a method of treating”, “a method of treatment” or its equivalent, when applied to, for example, cancer refers to a procedure or course of action that is designed to reduce or eliminate the number of cancer cells in a patient, or to alleviate the symptoms of a cancer. “A method of treating” cancer or another proliferative disorder does not necessarily mean that the cancer cells or other disorder will, in fact, be eliminated, that the number of cells or disorder will, in fact, be reduced, or that the symptoms of a cancer or other disorder will, in fact, be alleviated. Often, a method of treating cancer will be performed even with a low likelihood of success, but which, given the medical history and estimated survival expectancy of a patient, is nevertheless deemed to induce an overall beneficial course of action.

The terms “combination”, “co-administration” or “co-administering” refer to the administration of the BET inhibitor, the Bcl-2 inhibitor and the anti-CD20 antibody according to the invention in one or several formulations. The co-administration can be simultaneous or sequential in either order, wherein preferably there is a time period while two (or all) active agents simultaneously exert their biological activities. When the three therapeutic agents are co-administered sequentially, the can for example all be administered either on the same day in three separate administrations, or one of the agents can be administered on day 1 and the second and third can be co-administered on day 2 to day 7, or on day 2 to 4. Thus in one embodiment the term “sequentially” means within 7 days or 4 days after the dose of the first component; and the term “simultaneously” means at the same time or on the same day. The terms “co-administration” with respect to the maintenance doses of the anti-CD20 antibody, the Bcl-2 inhibitor and the BET inhibitor mean that the maintenance doses can be either co-administered simultaneously, if the treatment cycle is appropriate for all drugs, e.g. every week. Or the Bcl-2 inhibitor and the BET inhibitor can be administered e.g. every first to third day and the anti-CD20 antibody can be administered every week. Or the maintenance doses are co-administered sequentially, either within one or within several days.

It is self-evident that the antibodies and inhibitors are administered to the patient in a “therapeutically effective amount” (or simply “effective amount”) which is the amount of the respective compound or combination that will elicit the biological or medical response of a tissue, system, animal or human that is being sought by the researcher, veterinarian, medical doctor or other clinician.

The amount of co-administration of the BET inhibitor inhibitor, the Bcl-2 inhibitor and the anti-CD20 antibody and the timing of co-administration will depend on the type (species, gender, age, weight, etc.) and condition of the patient being treated and the severity of the disease or condition being treated.

The BET inhibitor is preferably administered subcutaneously.

The BET inhibitor is preferably administered at a dose between about 0.3 mg/kg/d and about 0.65 mg/kg/d.

The BET inhibitor is preferably administered daily for 14 consecutive days every 3 weeks (i.e. 2 weeks of dosing, 1 week of rest).

The BET inhibitor is preferably administered subcutaneously, at a dose between about 0.3 mg/kg/d and about 0.65 mg/kg/d.

The BET inhibitor is preferably administered subcutaneously, at a dose between about 0.3 mg/kg/d and about 0.65 mg/kg/d for 14 consecutive days every 3 weeks (i.e. 2 weeks of dosing, 1 week of rest).

The BET inhibitor is preferably RG6146.

The administration of the BET inhibitor, in particular RG6146, can be interrupted for up to 3 weeks, i.e 1, 2 or 3 weeks.

The Bcl-2 inhibitor is preferably administered orally.

The Bcl-2 inhibitor is preferably administered at a dose between about 400 mg/d to about 800 mg/d.

The Bcl-2 inhibitor is preferably administered orally, at a dose between about 400 mg/d and about 800 mg/d.

The Bcl-2 inhibitor is preferably administered daily (i.e. every day). This is called a continuous administration.

The Bcl-2 inhibitor is preferably daily administered orally, at a dose between about 400 mg/d and about 800 mg/d.

The Bcl-2 inhibitor is preferably venetoclax.

The anti-CD20 antibody is preferably administered intravenously.

The anti-CD20 antibody is preferably administered at a dose of about 375 mg/m² (body surface area dosing).

The anti-CD20 antibody is preferably administered weekly (i.e. once a week).

The anti-CD20 antibody is preferably administered intravenously, at a dose of about 375 mg/m² (body surface area dosing).

The anti-CD20 antibody is preferably weekly administered intravenously, at a dose of about 375 mg/m² (body surface area dosing), i.e. about 375 mg/m² once a week.

For example, for an adult of average size or body surface area, the dose of the anti-CD20 antibody can be about 10 mg/kg.

The anti-CD20 antibody is preferably rituximab or obinutuzumab, more preferably rituximab.

The administration cycles of the BET inhibitor, Bcl-2 inhibitor and anti-CD20 antibody are preferably initiated on the same day.

Depending on the type and severity of the disease, the following amounts can be administered: about 0.3 mg/kg/d to about 0.65 mg/kg/d of the BET inhibitor, preferably RG6146; about 400 mg/d to about 800 mg/d of the Bcl-2 inhibitor, preferably venetoclax; and about 375 mg/m² (body surface area dosing) of the anti-CD20 antibody, preferably rituximab.

A particular advantageous combination is about 0.3 mg/kg/d to about 0.65 mg/kg/d of the BET inhibitor, preferably RG6146, every day for 14 consecutive days every 3 weeks (i.e. 2 weeks of dosing, 1 week of rest); about 400 mg/d to about 800 mg/d continuously (i.e. every day) of the Bcl-2 inhibitor, preferably venetoclax; about 375 mg/m² (body surface area dosing) weekly (i.e. once a week) of the anti-CD20 antibody, preferably rituximab.

A further particular advantageous combination is about 0.3 mg/kg/d to about 0.65 mg/kg/d of the BET inhibitor, preferably RG6146, subcutaneously every day for 14 consecutive days every 3 weeks (i.e. 2 weeks of dosing, 1 week of rest); about 400 mg/d to about 800 mg/d continuously (i.e. every day) and orally of the Bcl-2 inhibitor, preferably venetoclax; about 375 mg/m² (body surface area dosing) weekly (i.e. once a week) and intravenously of the anti-CD20 antibody, preferably rituximab.

Alternatively, the anti-CD20 antibody, in particular the Type II anti-CD20 antibody, in particular obinutuzumab, can be administered in 6 cycles of 28 days as follows: about 1000 mg at days 1, 8 and 15 for cycle 1; about 1000 mg at day 1 for cycles 2-6.

Obinutuzumab is also preferably administered intravenously.

Depending on the type and severity of the disease, the following amounts can thus also be administered: about 0.3 mg/kg/d to about 0.65 mg/kg/d of the BET inhibitor, preferably RG6146; about 400 mg/d to about 800 mg/d of the Bcl-2 inhibitor, preferably venetoclax; and about 1000 mg at days 1, 8 and 15 of a 28 days cycle of the anti-CD20 antibody, preferably obinutuzumab.

A particular advantageous combination is about 0.3 mg/kg/d to about 0.65 mg/kg/d of the BET inhibitor, preferably RG6146, every day for 14 consecutive days every 3 weeks (i.e. 2 weeks of dosing, 1 week of rest); about 400 mg/d to about 800 mg/d continuously (i.e. every day) of the Bcl-2 inhibitor, preferably venetoclax; about 1000 mg at days 1, 8 and 15 for cycle 1 (28 days cycle) of the anti-CD20 antibody, preferably obinutuzumab and about 1000 mg at day 1 for cycles 2-6 (28 days cycle) of the anti-CD20 antibody, preferably obinutuzumab.

A further particular advantageous combination is about 0.3 mg/kg/d to about 0.65 mg/kg/d of the BET inhibitor, preferably RG6146, subcutaneously every day for 14 consecutive days every 3 weeks (i.e. 2 weeks of dosing, 1 week of rest); about 400 mg/d to about 800 mg/d continuously (i.e. every day) and orally of the Bcl-2 inhibitor, preferably venetoclax; about 1000 mg subcutaneously at days 1, 8 and 15 for cycle 1 (28 days cycle) of the anti-CD20 antibody, preferably obinutuzumab and about 1000 mg subcutaneously at day 1 for cycles 2-6 (28 days cycle) of the anti-CD20 antibody, preferably obinutuzumab.

In the above dosing regime, the administration of the BET inhibitor, in particular RG6146, can be interrupted for up to 3 weeks, i.e 1, 2 or 3 weeks.

In the above dosing regime, the administration of the Bcl-2 inhibitor, in particular venetoclax, can be interrupted for up to 3 weeks, i.e 1, 2 or 3 weeks.

The recommended dose may vary when there is a further co-administration of a chemotherapeutic agent.

The present invention is useful for preventing or reducing metastasis or further dissemination in such a patient suffering from DLBCL. This invention is useful for increasing the duration of survival of such a patient, increasing the progression free survival of such a patient, increasing the duration of response, resulting in a statistically significant and clinically meaningful improvement of the treated patient as measured by the duration of survival, progression free survival, response rate or duration of response. In a preferred embodiment, this invention is useful for increasing the response rate in a group of patients.

In the context of this invention, additional other cytotoxic, chemotherapeutic or anti-cancer agents, or compounds or ionizing radiation that enhance the effects of such agents (e.g. cytokines) may be used. Such molecules are suitably present in combination in amounts that are effective for the purpose intended.

Such additional agents include, for example: alkylating agents or agents with an alkylating action, such as cyclophosphamide (CTX; e.g. Cytoxan®), chlorambucil (CHL; e.g. Leukeran®), cisplatin (CisP; e.g. Platinol®) busulfan (e.g. Myleran®), melphalan, carmustine (BCNU), streptozotocin, triethylenemelamine (TEM), mitomycin C, and the like; anti-metabolites, such as methotrexate (MTX), etoposide (VP16; e.g. Vepesid®), 6-mercaptopurine (6MP), 6-thiocguanine (6TG), cytarabine (Ara-C), 5-fluorouracil (5-FU), capecitabine (e.g. Xeloda®), dacarbazine (DTIC), and the like; antibiotics, such as actinomycin D, doxorubicin (DXR; e.g. Adriamycin®), daunorubicin (daunomycin), bleomycin, mithramycin and the like; alkaloids, such as vinca alkaloids such as vincristine (VCR), vinblastine, and the like; and other antitumor agents, such as paclitaxel (e.g. Taxol®) and paclitaxel derivatives, the cytostatic agents, glucocorticoids such as dexamethasone (DEX; e.g. Decadron®) and corticosteroids such as prednisone, nucleoside enzyme inhibitors such as hydroxyurea, amino acid depleting enzymes such as asparaginase, leucovorin and other folic acid derivatives, and similar, diverse antitumor agents. The following agents may also be used as additional agents: arnifostine (e.g. Ethyol®), dactinomycin, mechlorethamine (nitrogen mustard), streptozocin, cyclophosphamide, lomustine (CCNU), doxorubicin lipo (e.g. Doxil®), gemcitabine (e.g. Gemzar®), daunorubicin lipo (e.g. Daunoxome®), procarbazine, mitomycin, docetaxel (e.g. Taxotere®), aldesleukin, carboplatin, oxaliplatin, cladribine, camptothecin, CPT 11 (irinotecan), 10-hydroxy 7-ethyl-camptothecin (SN38), floxuridine, fludarabine, ifosfamide, idarubicin, mesna, interferon beta, interferon alpha, mitoxantrone, topotecan, leuprolide, megestrol, melphalan, mercaptopurine, plicamycin, mitotane, pegaspargase, pentostatin, pipobroman, plicamycin, tamoxifen, teniposide, testolactone, thioguanine, thiotepa, uracil mustard, vinorelbine or chlorambucil.

The use of the cytotoxic and anticancer agents described above as well as antiproliferative target-specific anticancer drugs like protein kinase inhibitors in chemotherapeutic regimens is generally well characterized in the cancer therapy arts, and their use herein falls under the same considerations for monitoring tolerance and effectiveness and for controlling administration routes and dosages, with some adjustments. For example, the actual dosages of the cytotoxic agents may vary depending upon the patient's cultured cell response determined by using histoculture methods. Generally, the dosage will be reduced compared to the amount used in the absence of additional other agents.

Typical dosages of an effective cytotoxic agent can be in the ranges recommended by the manufacturer, and where indicated by in vitro responses or responses in animal models, can be reduced by up to about one order of magnitude concentration or amount. Thus, the actual dosage will depend upon the judgment of the physician, the condition of the patient, and the effectiveness of the therapeutic method based on the in vitro responsiveness of the primary cultured malignant cells or histocultured tissue sample, or the responses observed in the appropriate animal models.

In the context of this invention, an effective amount of ionizing radiation may be carried out and/or a radiopharmaceutical may be used. The source of radiation can be either external or internal to the patient being treated. When the source is external to the patient, the therapy is known as external beam radiation therapy (EBRT). When the source of radiation is internal to the patient, the treatment is called brachytherapy (BT). Radioactive atoms for use in the context of this invention can be selected from the group including, but not limited to, radium, yttrium-90, cesium-137, iridium-192, americium-241, gold-198, cobalt-57, copper-67, technetium-99, iodine-123, iodine-131, and indium-111. Is also possible to label the antibody with such radioactive isotopes.

Radiation therapy is a standard treatment for controlling unresectable or inoperable tumors and/or tumor metastases. Improved results have been seen when radiation therapy has been combined with chemotherapy. Radiation therapy is based on the principle that high-dose radiation delivered to a target area will result in the death of reproductive cells in both tumor and normal tissues. The radiation dosage regimen is generally defined in terms of radiation absorbed dose (Gy), time and fractionation, and must be carefully defined by the oncologist. The amount of radiation a patient receives will depend on various considerations, but the two most important are the location of the tumor in relation to other critical structures or organs of the body, and the extent to which the tumor has spread. A typical course of treatment for a patient undergoing radiation therapy will be a treatment schedule over a 1 to 6 week period, with a total dose of between 10 and 80 Gy administered to the patient in a single daily fraction of about 1.8 to 2.0 Gy, 5 days a week. In a preferred embodiment of this invention there is synergy when tumors in human patients are treated with the combination treatment of the invention and radiation. In other words, the inhibition of tumor growth by means of the agents comprising the combination of the invention is enhanced when combined with radiation, optionally with additional chemotherapeutic or anticancer agents. Parameters of adjuvant radiation therapies are, for example, contained in WO 99/60023.

As used herein, a “pharmaceutically acceptable carrier” or “pharmaceutically acceptable excipient” is intended to include any and all material compatible with pharmaceutical administration including solvents, dispersion media, coatings, antibacterial and antifungal agents, isotonic and absorption delaying agents, and other materials and compounds compatible with pharmaceutical administration. Except insofar as any conventional media or agent is incompatible with the active compound, use thereof in the compositions of the invention is contemplated. Supplementary active compounds can also be incorporated into the compositions.

Pharmaceutical compositions can be obtained by processing the BET inhibitor inhibitor, the Bcl-2 inhibitor and the anti-CD20 antibody according to this invention with pharmaceutically acceptable, inorganic or organic carriers or excipients. Lactose, corn starch or derivatives thereof, talc, stearic acids or it's salts and the like can be used, for example, as such carriers for tablets, coated tablets, dragees and hard gelatine capsules. Suitable carriers for soft gelatine capsules are, for example, vegetable oils, waxes, fats, semi-solid and liquid polyols and the like. Depending on the nature of the active substance no carriers are, however, usually required in the case of soft gelatine capsules. Suitable carriers for the production of solutions and syrups are, for example, water, polyols, glycerol, vegetable oil and the like. Suitable carriers for suppositories are, for example, natural or hardened oils, waxes, fats, semi-liquid or liquid polyols and the like.

The pharmaceutical compositions can, moreover, contain preservatives, solubilizers, stabilizers, wetting agents, emulsifiers, sweeteners, colorants, flavorants, salts for varying the osmotic pressure, buffers, masking agents or antioxidants. They can also contain still other therapeutically valuable substances.

Pharmaceutical compositions of the anti-CD20 antibody alone can be prepared for storage by mixing an antibody having the desired degree of purity with optional pharmaceutically acceptable carriers, excipients or stabilizers (Remington's Pharmaceutical Sciences 16th edition, Osol, A. (ed.) (1980)), in the form of lyophilized formulations or aqueous solutions. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEEN™, PLURONICS™ or polyethylene glycol (PEG).

Pharmaceutical compositions of the BET inhibitor and of the Bcl-2 inhibitor include those suitable for oral, nasal, topical (including buccal and sublingual), rectal, vaginal and/or parenteral administration. The compositions may conveniently be presented in unit dosage form and may be prepared by any methods well known in the art of pharmacy. The amount of active ingredient which can be combined with a carrier material to produce a single dosage form will vary depending upon the host being treated, as well as the particular mode of administration. The amount of active ingredient which can be combined with a carrier material to produce a single dosage form will generally be that amount of a Bcl-2 inhibitor or a BET inhibitor which produces a therapeutic effect. Generally, out of one hundred percent, this amount will range from about 1 percent to about 90 percent of active ingredient, preferably from about 5 percent to about 70 percent, most preferably from about 10 percent to about 30 percent. Methods of preparing these compositions include the step of bringing into association a Bcl-2 inhibitor or a BET inhibitor with the carrier and, optionally, one or more accessory ingredients. In general, the pharmaceutical compositions can be prepared by uniformaly and intimately bringing into association a Bcl-2 inhibitor or a BET inhibitor with liquid carriers, or finely divided solid carriers, or both, and then, if necessary, shaping the product. Pharmaceutical compositions suitable for oral administration may be in the form of capsules, cachets, sachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a Bcl-2 inhibitor or a BET inhibitor as an active ingredient. A Bcl-2 inhibitor and a BET inhibitor may also be administered as a bolus, electuary or paste.

In further embodiments of the invention, the BET inhibitor inhibitor, the Bcl-2 inhibitor and the anti-CD20 antibody are formulated into one, two or three separate pharmaceutical compositions.

The active ingredients may also be entrapped in microcapsules prepared, for example, by coacervation techniques or by interracial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences, 16th edition, Osol, A. (ed.) (1980).

Sustained-release preparations may be prepared. Suitable examples of sustained-release preparations include semipermeable matrices of solid hydrophobic polymers containing the antibody, which matrices are in the form of shaped articles, e.g. films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and gamma-ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid.

The formulations to be used for in vivo administration must be sterile. This is readily accomplished by filtration through sterile filtration membranes.

Sequence Listings
SEQ ID NO: 1 amino acid sequence of variable region of the heavy chain (VH) of murine monoclonal anti-CD20 antibody B-Ly1.
SEQ ID NO: 2 amino acid sequence of variable region of the light chain (VL) of murine monoclonal anti-CD20 antibody B-Ly1.
SEQ ID NO: 3-19 amino acid sequences of variable region of the heavy chain (VH) of humanized B-Ly1 antibodies (B-HH2 to B-HH9, B-HL8, and B-HL10 to B-HL17).
SEQ ID NO: 20 amino acid sequence of variable region of the light chain (VL) of humanized B-Ly1 antibody B-KV1.

The following examples and figures are provided to illustrate the invention and have no limiting character.

### EXAMPLE

### Example 1: In Vivo Antitumor Efficacy

The in vivo antitumor efficacy of the CD20 specific antibody obinutuzumab or rituximab in combination with Bcl-2 inhibitor venetoclax (GDC-0199) and BET inhibitor RG6146 was evaluated against WSU-DLCL2 xenografts (CD20+).

Test Agents

CD20 antibody obinutuzumab or rituximab was provided as stock solution from Roche, Basel, Switzerland. Antibody buffer included histidine. Antibody solution was diluted appropriately in buffer from stock prior injections. BET inhibitor RG6146 was provided as powder from Roche, Basel, Switzerland and resuspended prior to use. Bcl-2 inhibitor GDC-0199 was provided by Genentech, South San Francisco, USA and formulated prior to use.

Cell Line and Culture Conditions

The original WSU-DLCL2 human B cell NHL cell line (DLBCL) was purchased from DSMZ (Braunschweig, Germany). Expansion of tumor cells for the transplantation was done by the TAP CompacT CellBase Cell Culture Roboter according to the protocol. Tumor cell line was routinely cultured in RPMI 1640 medium, FCS 10% and L-Glutamin 2 mM at 37° C. in a water-saturated atmosphere at 5% CO₂. Culture passage was performed with trypsin/EDTA 1× splitting twice/week and passage 3 used for transplantation.

Animals

Female SCID beige mice, age 6-7 weeks at arrival, maintained under specific-pathogen-free condition with daily cycles of 12 h light/12 h darkness according to committed guidelines. Experimental study protocol was reviewed and approved by local government. After arrival animals were maintained in animal facility for one week to get accustomed to new environment and for observation. Continuous health monitoring was carried out on regular basis. Diet food and autoclaved water were provided ad libitum.

Monitoring

Animals were controlled daily for clinical symptoms and detection of adverse effects. For monitoring throughout the experiment body weight of animals was documented.

Treatment of Animals

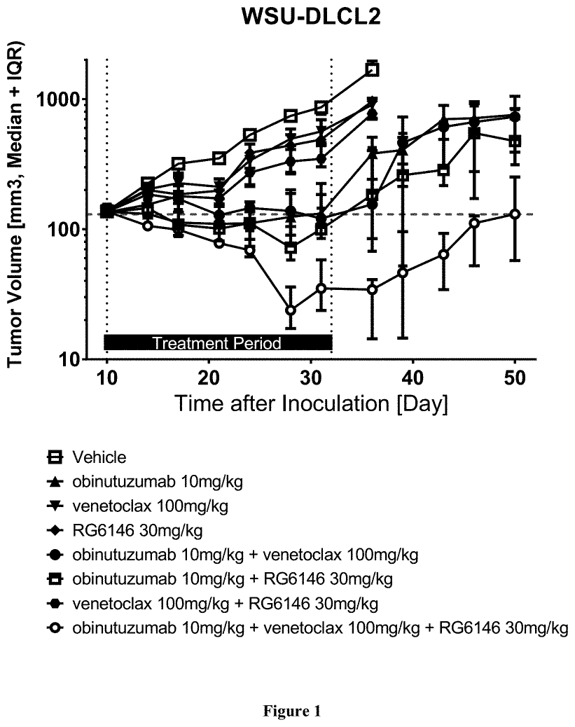

Animal treatment for study displayed in FIG. 1 started after randomisation when median tumor size was about 150 mm³. CD20 antibody obinutuzumab was administered as single agent and in combination at 10 mg/kg ip once weekly on days 10, 17, 24 and 31. The corresponding vehicle was administered on the same days. BET inhibitor RG6146 ip treatment at 30 mg/kg was done as single agent and in combination on days 10-18, 21-25 and 28-32. Finally, Bcl-2 inhibitor venetoclax (GDC-0199) was given orally at 100 mg/kg on days 10-18, 21-25 and 28-32 as single agent and in combination.

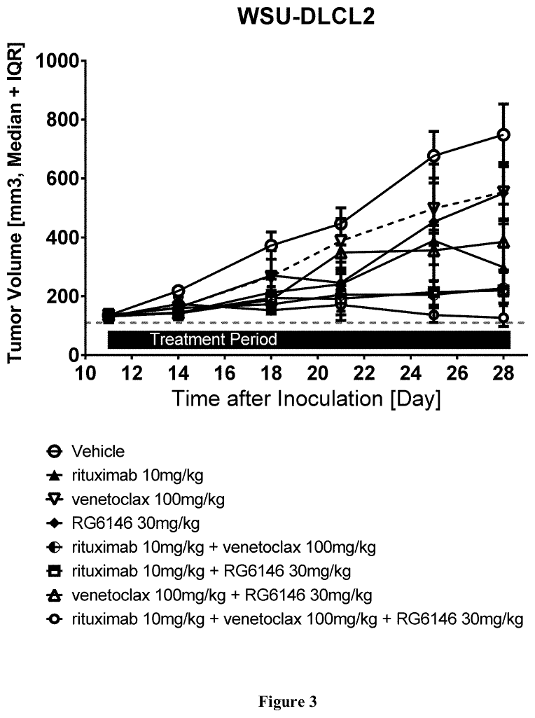

Animal treatment started in the study displayed in FIG. 3 after randomisation when median tumor size was about 130 mm³. CD20 antibody rituximab was administered as single agent and in combination at 10 mg/kg ip once weekly on days 10, 17 and 24. The corresponding vehicle was administered on the same days. BET inhibitor RG6146 ip treatment at 30 mg/kg was done as single agent and in combination on days 11-17. Finally, bcl-2 inhibitor venetoclax (GDC-0199) was given orally at 100 mg/kg on days 11-17 as single agent and in combination.

Antitumor Efficacy

For the study shown in FIG. 1, WSU-DLCL2 human Diffuse Large B-cell Lymphoma (DLBCL) cells (CD20+) were s.c. inoculated with Matrigel onto female SCID beige mice. Tumor bearing mice were randomized 10 days later to the indicated study groups and compound treatment initiated. Tumor bearing animals were treated with vehicle control, with the BET inhibitor RG6146 at 30 mg/kg, with the anti-CD20 antibody obinutuzumab at 10 mg/kg or with Bcl-2 inhibitor venetoclax (GDC-0199) at 100 mg/kg as single agent. Additionally, three groups were treated with a dual combination of either RG6146 and venetoclax, or RG6146 and obinutuzumab or obinutuzumab and venetoclax. Finally, one study group received the triple combination of BET inhibitor RG6146, CD20 antibody obinutuzumab and Bcl-2 inhibitor venetoclax (GDC-0199). As a result, all compounds given as single agent demonstrated significant anti-tumor efficacy against WSU-DLCL2 xenografts. In more detail treatment with the BET inhibitor RG6146 resulted in 46% tumor growth inhibition (TGI) against WSU-DLCL2 xenografts compared to control. A similar efficacy was noticed after treatment with the Bcl-2 inhibitor venetoclax (49% TGI), whereas the strongest efficacy as single agent was achieved after treatment with the anti-CD20 antibody obinutuzumab (TGI 84%). However, superior efficacy was observed for the triple combination group including the BET inhibitor RG6146 plus CD20 antibody obinutuzumab plus Bcl-2 inhibitor venetoclax. In more detail the triple combination approach substantially induced tumor regression which reached finally 75% with 22% complete tumor remissions. In constrast to this, the respective dual combination study arms were less efficacious and translated into tumor stasis (TGI about 100%). Notably, in the study follow-up, after treatment was discontinued, a substantial 2-fold delay in tumor regrowth was observed for the triple combination after 40 days. In constrast to this, the respective double combination regimens reached a tumor regrowth after about 20 days.

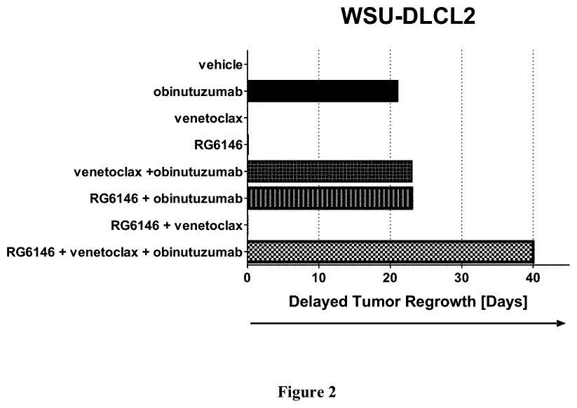

The results are illustrated in FIGS. 1-2 and Tables 2-4.

TABLE 2
Efficacy of RG6146, venetoclax and obinutuzumab (Day 10-50)

| Day | 10 | 14 | 17 | 21 | 24 | 28 | 31 | 36 | 39 | 43 | 46 | 50 |
|---|---|---|---|---|---|---|---|---|---|---|---|---|
| vehicle | 137 | 224 | 319 | 351 | 531 | 742 | 864 | 1679 | | | | |
| obinutuzumab 10 mg/kg | 136 | 131 | 112 | 110 | 111 | 124 | 132 | 382 | 405 | 700 | 713 | 758 |
| venetoclax 100 mg/kg | 137 | 202 | 226 | 212 | 337 | 494 | 565 | 906 | | | | |
| RG6146 30 mg/kg | 137 | 200 | 185 | 198 | 387 | 441 | 489 | 962 | | | | |
| obinutuzumab 10 mg/kg + | 136 | 185 | 171 | 128 | 145 | 138 | 121 | 155 | 462 | 611 | 667 | 731 |
| venetoclax 100 mg/kg | | | | | | | | | | | | |
| obinutuzumab 10 mg/kg + | 135 | 145 | 108 | 101 | 111 | 72 | 100 | 184 | 260 | 286 | 548 | 476 |
| RG6146 30 mg/kg | | | | | | | | | | | | |
| venetoclax 100 mg/kg + | 135 | 155 | 180 | 173 | 272 | 332 | 348 | 774 | | | | |
| RG6146 30 mg/kg | | | | | | | | | | | | |
| obinutuzumab 10 mg/kg + | 137 | 106 | 99 | 78 | 69 | 24 | 35 | 34 | 46 | 64 | 111 | 131 |
| venetoclax 100 mg/kg + | | | | | | | | | | | | |
| RG6146 30 mg/kg | | | | | | | | | | | | |

Table 2 lists the median tumor volume data plotted in FIG. 1.

TABLE 3
Efficacy of RG6146, venetoclax and obinutuzumab (Day 36)

| | Dose | | TGI | Regression | npTCR | Tumor |
|---|---|---|---|---|---|---|
| Compound | (mg/kg) | Schedule | % | % | and CI | free mice |
| control | — | iv | — | — | — | 0/10 |
| obinutuzumab | 10 | iv | 84 | — | 0.20 | 0/10 |
| | | | | | (CI 0.11- | |
| | | | | | 0.33) | |
| venetoclax | 100 | po | 49 | — | 0.53 | 0/10 |
| | | | | | (CI 0.32- | |
| | | | | | 0.71) | |
| RG6146 | 30 | ip | 46 | — | 0.50 | 0/9 |
| | | | | | (CI 0.32- | |
| | | | | | 0.76) | |
| obinutuzumab + | 10 | iv | >100 | 2 | 0.08 | 0/10 |
| venetoclax | 100 | po | | | (CI 0.03- | |
| | | | | | 0.30) | |
| obinutuzumab + | 10 | iv | 97 | 0 | 0.10 | 2/9 |
| RG6146 | 30 | ip | | | (CI 0.00- | |
| | | | | | 0.28) | |
| venetoclax + | 100 | po | 58 | — | 0.45 | 0/10 |
| RG6146 | 30 | ip | | | (CI 0.31- | |
| | | | | | 0.56) | |
| obinutuzumab + | 10 | iv | >100 | 75 | 0.02 | 2/9 |
| venetoclax + | 100 | po | | | (CI 0.00- | |
| RG6146 | 30 | ip | | | 0.04) | |

TCR: Treatment to Control Ratio;
pTCR: non-parametric Tumor Control Ratio;
CI: Confidence Interval

TABLE 4
Tumor growth delay (until Day 10-50)

| | Dose | | Tumor growth delay* |
|---|---|---|---|
| Compound | (mg/kg) | Schedule | (Days) |
| obinutuzumab | 10 | iv | 21 |
| obinutuzumab + | 10 | iv | 26 |
| venetoclax | 100 | po | |
| obinutuzumab + | 10 | iv | 26 |
| RG6146 | 30 | ip | |
| obinutuzumab + | 10 | iv | 40 |
| venetoclax + | 100 | po | |
| RG6146 | 30 | ip | |

*The tumor growth delay is the time (number of days) until relative tumor volume reached again 100% (100% at beginning, Day 10).

TABLE 5
Efficacy of RG6146, venetoclax and rituximab (Day 11-28)

| Day | 11 | 14 | 18 | 21 | 25 | 28 |
|---|---|---|---|---|---|---|
| vehicle | 135 | 218 | 372 | 446 | 677 | 749 |
| rituximab 10 mg/kg | 131 | 143 | 212 | 241 | 389 | 298 |
| venetoclax 100 mg/kg | 136 | 162 | 267 | 388 | 497 | 554 |
| RG6146 30 mg/kg | 134 | 161 | 270 | 245 | 453 | 550 |
| rituximab 10 mg/kg + | 135 | 160 | 172 | 205 | 205 | 227 |
| venetoclax 100 mg/kg | | | | | | |
| rituximab 10 mg/kg + | 134 | 145 | 194 | 191 | 214 | 219 |
| RG6146 30 mg/kg | | | | | | |
| venetoclax 100 mg/kg + | 136 | 141 | 188 | 348 | 355 | 385 |
| RG6146 30 mg/kg | | | | | | |
| rituximab 10 mg/kg + | 131 | 174 | 152 | 170 | 136 | 126 |
| venetoclax 100 mg/kg + | | | | | | |
| RG6146 30 mg/kg | | | | | | |

Table 5 lists the median tumor volume data plotted in FIG. 3

TABLE 6
Efficacy of RG6146, venetoclax and rituximab (Day 28)

| | Dose | | TGI | npTCR | Tumor |
|---|---|---|---|---|---|
| Compound | (mg/kg)) | Schedule | % | and CI | free mice |
| control | — | iv | — | — | 0/10 |
| rituximab | 10 | iv | 75 | 0.26 | 0/10 |
| | | | | (CI 0.09- | |
| | | | | 0.60) | |
| venetoclax | 100 | po | 34 | 0.66 | 0/10 |
| | | | | (CI 0.45- | |
| | | | | 0.96) | |
| RG6146 | 30 | ip | 30 | 0.71 | 0/9 |
| | | | | (CI 0.50- | |
| | | | | 1.00) | |
| rituximab + | 10 | iv | 87 | 0.13 | 0/10 |
| venetoclax | 100 | po | | (CI 0.06- | |
| | | | | 0.34) | |
| rituximab + | 10 | iv | 83 | 0.19 | 0/9 |
| RG6146 | 30 | ip | | (CI 0.00- | |
| | | | | 0.36) | |
| venetoclax + | 100 | po | 56 | 0.42 | 0/10 |
| RG6146 | 30 | ip | | (CI 0.25- | |
| | | | | 0.64) | |
| rituximab + | 10 | iv | >100 | −0.04 | 1/10 |
| venetoclax + | 100 | po | | (CI −0.10- | |
| RG6146 | 30 | ip | | 0.27) | |

TCR: Treatment to Control Ratio;
pTCR: non-parametric Tumor Control Ratio;
CI: Confidence Interval

As disclosed herein and also appended in the sequence listing, the following sequences are part of the present invention:

Sequences
amino acid sequence of variable region of the heavy chain (VH) of murine
monoclonal anti-CD20 antibody B-Ly1
SEQ ID NO: 1
Gly Pro Glu Leu Val Lys Pro Gly Ala Ser Val Lys Ile Ser Cys Lys
Ala Ser Gly Tyr Ala Phe Ser Tyr Ser Trp Met Asn Trp Val Lys Leu
Arg Pro Gly Gln Gly Leu Glu Trp Ile Gly Arg Ile Phe Pro Gly Asp
Gly Asp Thr Asp Tyr Asn Gly Lys Phe Lys Gly Lys Ala Thr Leu Thr
Ala Asp Lys Ser Ser Asn Thr Ala Tyr Met Gln Leu Thr Ser Leu Thr
Ser Val Asp Ser Ala Val Tyr Leu Cys Ala Arg Asn Val Phe Asp Gly
Tyr Trp Leu Val Tyr Trp Gly Gln Gly Thr Leu Val Thr Val Ser Ala
amino acid sequence of variable region of the light chain (VL) of murine
monoclonal anti-CD20 antibody B-Ly1
SEQ ID NO: 2
Asn Pro Val Thr Leu Gly Thr Ser Ala Ser Ile Ser Cys Arg Ser Ser
Lys Ser Leu Leu His Ser Asn Gly Ile Thr Tyr Leu Tyr Trp Tyr Leu
Gln Lys Pro Gly Gln Ser Pro Gln Leu Leu Ile Tyr Gln Met Ser Asn
Leu Val Ser Gly Val Pro Asp Arg Phe Ser Ser Ser Gly Ser Gly Thr
Asp Phe Thr Leu Arg Ile Ser Arg Val Glu Ala Glu Asp Val Gly Val
Tyr Tyr Cys Ala Gln Asn Leu Glu Leu Pro Tyr Thr Phe Gly Gly Gly
Thr Lys Leu Glu Ile Lys Arg
amino acid sequences of variable region of the heavy chain (VH) of humanized
B-Ly1 antibody (B-HH2)
SEQ ID NO: 3
Gln Val Gln Leu Val Gln Ser Gly Ala Glu Val Lys Lys Pro Gly Ser
Ser Val Lys Val Ser Cys Lys Ala Ser Gly Tyr Ala Phe Ser Tyr Ser
Trp Met Asn Trp Val Arg Gln Ala Pro Gly Gln Gly Leu Glu Trp Met
Gly Arg Ile Phe Pro Gly Asp Gly Asp Thr Asp Tyr Asn Gly Lys Phe
Lys Gly Arg Val Thr Ile Thr Ala Asp Lys Ser Thr Ser Thr Ala Tyr
Met Glu Leu Ser Ser Leu Arg Ser Glu Asp Thr Ala Val Tyr Tyr Cys
Ala Arg Asn Val Phe Asp Gly Tyr Trp Leu Val Tyr Trp Gly Gln Gly
Thr Leu Val Thr Val Ser Ser
amino acid sequences of variable region of the heavy chain (VH) of humanized
B-Ly1 antibody (B-HH3)
SEQ ID NO: 4
Gln Val Gln Leu Val Gln Ser Gly Ala Glu Val Lys Lys Pro Gly Ser
Ser Val Lys Val Ser Cys Lys Ala Ser Gly Tyr Ala Phe Ser Tyr Ser
Trp Met Asn Trp Val Arg Gln Ala Pro Gly Gln Gly Leu Glu Trp Met
Gly Arg Ile Phe Pro Gly Asp Gly Asp Thr Asp Tyr Asn Gly Lys Phe
Lys Gly Arg Val Thr Ile Thr Ala Asp Lys Ser Thr Ser Thr Ala Tyr
Met Glu Leu Ser Ser Leu Arg Ser Glu Asp Thr Ala Val Tyr Leu Cys
Ala Arg Asn Val Phe Asp Gly Tyr Trp Leu Val Tyr Trp Gly Gln Gly
Thr Leu Val Thr Val Ser Ser
amino acid sequences of variable region of the heavy chain (VH) of humanized
B-Ly1 antibody (B-HH4)
SEQ ID NO: 5
Gln Val Gln Leu Val Gln Ser Gly Ala Glu Val Lys Lys Pro Gly Ala
Ser Val Lys Val Ser Cys Lys Val Ser Gly Tyr Ala Phe Ser Tyr Ser
Trp Met Asn Trp Val Arg Gln Ala Pro Gly Gln Gly Leu Glu Trp Met
Gly Arg Ile Phe Pro Gly Asp Gly Asp Thr Asp Tyr Asn Gly Lys Phe
Lys Gly Arg Val Thr Ile Thr Ala Asp Lys Ser Thr Ser Thr Ala Tyr
Met Glu Leu Ser Ser Leu Arg Ser Glu Asp Thr Ala Val Tyr Tyr Cys
Ala Arg Asn Val Phe Asp Gly Tyr Trp Leu Val Tyr Trp Gly Gln Gly
Thr Leu Val Thr Val Ser Ser
amino acid sequences of variable region of the heavy chain (VH) of humanized
B-Ly1 antibody (B-HH5)
SEQ ID NO: 6
Gln Val Gln Leu Val Gln Ser Gly Ala Glu Val Lys Lys Pro Gly Ser
Ser Val Lys Val Ser Cys Lys Ala Ser Gly Tyr Ala Phe Ser Tyr Ser
Trp Met Ser Trp Val Arg Gln Ala Pro Gly Gln Gly Leu Glu Trp Met
Gly Arg Ile Phe Pro Gly Asp Gly Asp Thr Asp Tyr Asn Gly Lys Phe
Lys Gly Arg Val Thr Ile Thr Ala Asp Lys Ser Thr Ser Thr Ala Tyr
Met Glu Leu Ser Ser Leu Arg Ser Glu Asp Thr Ala Val Tyr Tyr Cys
Ala Arg Asn Val Phe Asp Gly Tyr Trp Leu Val Tyr Trp Gly Gln Gly
Thr Leu Val Thr Val Ser Ser
amino acid sequences of variable region of the heavy chain (VH) of humanized
B-Ly1 antibody (B-HH6)
SEQ ID NO: 7
Gln Val Gln Leu Val Gln Ser Gly Ala Glu Val Lys Lys Pro Gly Ser
Ser Val Lys Val Ser Cys Lys Ala Ser Gly Tyr Ala Phe Ser Tyr Ser
Trp Ile Asn Trp Val Arg Gln Ala Pro Gly Gln Gly Leu Glu Trp Met
Gly Arg Ile Phe Pro Gly Asp Gly Asp Thr Asp Tyr Asn Gly Lys Phe
Lys Gly Arg Val Thr Ile Thr Ala Asp Lys Ser Thr Ser Thr Ala Tyr
Met Glu Leu Ser Ser Leu Arg Ser Glu Asp Thr Ala Val Tyr Tyr Cys
Ala Arg Asn Val Phe Asp Gly Tyr Trp Leu Val Tyr Trp Gly Gln Gly
Thr Leu Val Thr Val Ser Ser
amino acid sequences of variable region of the heavy chain (VH) of humanized
B-Ly1 antibody (B-HH7)
SEQ ID NO: 8
Gln Val Gln Leu Val Gln Ser Gly Ala Glu Val Lys Lys Pro Gly Ser
Ser Val Lys Val Ser Cys Lys Ala Ser Gly Tyr Ala Phe Ser Tyr Ser
Trp Ile Ser Trp Val Arg Gln Ala Pro Gly Gln Gly Leu Glu Trp Met
Gly Arg Ile Phe Pro Gly Asp Gly Asp Thr Asp Tyr Asn Gly Lys Phe
Lys Gly Arg Val Thr Ile Thr Ala Asp Lys Ser Thr Ser Thr Ala Tyr
Met Glu Leu Ser Ser Leu Arg Ser Glu Asp Thr Ala Val Tyr Tyr Cys
Ala Arg Asn Val Phe Asp Gly Tyr Trp Leu Val Tyr Trp Gly Gln Gly
Thr Leu Val Thr Val Ser Ser
amino acid sequences of variable region of the heavy chain (VH) of humanized
B-Ly1 antibody (B-HH8)
SEQ ID NO: 9
Gln Val Gln Leu Val Gln Ser Gly Ala Glu Val Lys Lys Pro Gly Ala
Ser Val Lys Val Ser Cys Lys Ala Ser Gly Tyr Thr Phe Thr Tyr Ser
Trp Met Asn Trp Val Arg Gln Ala Pro Gly Gln Gly Leu Glu Trp Met
Gly Arg Ile Phe Pro Gly Asp Gly Asp Thr Asp Tyr Asn Gly Lys Phe
Lys Gly Arg Val Thr Ile Thr Ala Asp Lys Ser Thr Ser Thr Ala Tyr
Met Glu Leu Ser Ser Leu Arg Ser Glu Asp Thr Ala Val Tyr Tyr Cys
Ala Arg Asn Val Phe Asp Gly Tyr Trp Leu Val Tyr Trp Gly Gln Gly
Thr Leu Val Thr Val Ser Ser
amino acid sequences of variable region of the heavy chain (VH) of humanized
B-Ly1 antibody (B-HH9)
SEQ ID NO: 10
Gln Val Gln Leu Val Gln Ser Gly Ala Glu Val Lys Lys Pro Gly Ala
Ser Val Lys Val Ser Cys Lys Ala Ser Gly Tyr Thr Phe Ser Tyr Ser
Trp Met Asn Trp Val Arg Gln Ala Pro Gly Gln Gly Leu Glu Trp Met
Gly Arg Ile Phe Pro Gly Asp Gly Asp Thr Asp Tyr Asn Gly Lys Phe
Lys Gly Arg Val Thr Ile Thr Ala Asp Lys Ser Thr Ser Thr Ala Tyr
Met Glu Leu Ser Ser Leu Arg Ser Glu Asp Thr Ala Val Tyr Tyr Cys
Ala Arg Asn Val Phe Asp Gly Tyr Trp Leu Val Tyr Trp Gly Gln Gly
Thr Leu Val Thr Val Ser Ser
amino acid sequences of variable region of the heavy chain (VH) of humanized
B-Ly1 antibody (B-HL8)
SEQ ID NO: 11
Glu Val Gln Leu Val Glu Ser Gly Gly Gly Leu Val Lys Pro Gly Gly
Ser Leu Arg Leu Ser Cys Ala Ala Ser Gly Phe Thr Phe Ser Tyr Ser
Trp Met Asn Trp Val Arg Gln Ala Pro Gly Lys Gly Leu Glu Trp Val
Gly Arg Ile Phe Pro Gly Asp Gly Asp Thr Asp Tyr Asn Gly Lys Phe
Lys Gly Arg Val Thr Ile Thr Ala Asp Lys Ser Thr Ser Thr Ala Tyr
Met Glu Leu Ser Ser Leu Arg Ser Glu Asp Thr Ala Val Tyr Tyr Cys
Ala Arg Asn Val Phe Asp Gly Tyr Trp Leu Val Tyr Trp Gly Gln Gly
Thr Leu Val Thr Val Ser Ser
amino acid sequences of variable region of the heavy chain (VH) of humanized
B-Ly1 antibody (B-HL10)
SEQ ID NO: 12
Glu Val Gln Leu Val Glu Ser Gly Gly Gly Leu Val Lys Pro Gly Gly
Ser Leu Arg Leu Ser Cys Ala Ala Ser Gly Phe Ala Phe Ser Tyr Ser
Trp Met Asn Trp Val Arg Gln Ala Pro Gly Lys Gly Leu Glu Trp Val
Gly Arg Ile Phe Pro Gly Asp Gly Asp Thr Asp Tyr Asn Gly Lys Phe
Lys Gly Arg Val Thr Ile Thr Ala Asp Lys Ser Thr Ser Thr Ala Tyr
Met Glu Leu Ser Ser Leu Arg Ser Glu Asp Thr Ala Val Tyr Tyr Cys
Ala Arg Asn Val Phe Asp Gly Tyr Trp Leu Val Tyr Trp Gly Gln Gly
Thr Leu Val Thr Val Ser Ser
amino acid sequences of variable region of the heavy chain (VH) of humanized
B-Ly1 antibody (B-HL11)
SEQ ID NO: 13
Gln Val Gln Leu Val Glu Ser Gly Gly Gly Leu Val Lys Pro Gly Gly
Ser Leu Arg Leu Ser Cys Ala Ala Ser Gly Phe Thr Phe Ser Tyr Ser
Trp Met Asn Trp Val Arg Gln Ala Pro Gly Lys Gly Leu Glu Trp Val
Gly Arg Ile Phe Pro Gly Asp Gly Asp Thr Asp Tyr Asn Gly Lys Phe
Lys Gly Arg Val Thr Ile Thr Ala Asp Lys Ser Thr Ser Thr Ala Tyr
Met Glu Leu Ser Ser Leu Arg Ser Glu Asp Thr Ala Val Tyr Tyr Cys
Ala Arg Asn Val Phe Asp Gly Tyr Trp Leu Val Tyr Trp Gly Gln Gly
Thr Leu Val Thr Val Ser Ser
amino acid sequences of variable region of the heavy chain (VH) of humanized
B-Ly1 antibody (B-HL12)
SEQ ID NO: 14
Glu Val Gln Leu Val Glu Ser Gly Ala Gly Leu Val Lys Pro Gly Gly
Ser Leu Arg Leu Ser Cys Ala Ala Ser Gly Phe Thr Phe Ser Tyr Ser
Trp Met Asn Trp Val Arg Gln Ala Pro Gly Lys Gly Leu Glu Trp Met
Gly Arg Ile Phe Pro Gly Asp Gly Asp Thr Asp Tyr Asn Gly Lys Phe
Lys Gly Arg Val Thr Ile Thr Ala Asp Lys Ser Thr Ser Thr Ala Tyr
Met Glu Leu Ser Ser Leu Arg Ser Glu Asp Thr Ala Val Tyr Tyr Cys
Ala Arg Asn Val Phe Asp Gly Tyr Trp Leu Val Tyr Trp Gly Gln Gly
Thr Leu Val Thr Val Ser Ser
amino acid sequences of variable region of the heavy chain (VH) of humanized
B-Ly1 antibody (B-HL13)
SEQ ID NO: 15
Glu Val Gln Leu Val Glu Ser Gly Gly Gly Val Val Lys Pro Gly Gly
Ser Leu Arg Leu Ser Cys Ala Ala Ser Gly Phe Thr Phe Ser Tyr Ser
Trp Met Asn Trp Val Arg Gln Ala Pro Gly Lys Gly Leu Glu Trp Met
Gly Arg Ile Phe Pro Gly Asp Gly Asp Thr Asp Tyr Asn Gly Lys Phe
Lys Gly Arg Val Thr Ile Thr Ala Asp Lys Ser Thr Ser Thr Ala Tyr
Met Glu Leu Ser Ser Leu Arg Ser Glu Asp Thr Ala Val Tyr Tyr Cys
Ala Arg Asn Val Phe Asp Gly Tyr Trp Leu Val Tyr Trp Gly Gln Gly
Thr Leu Val Thr Val Ser Ser
amino acid sequences of variable region of the heavy chain (VH) of humanized
B-Ly1 antibody (B-HL14)
SEQ ID NO: 16
Glu Val Gln Leu Val Glu Ser Gly Gly Gly Leu Lys Lys Pro Gly Gly
Ser Leu Arg Leu Ser Cys Ala Ala Ser Gly Phe Thr Phe Ser Tyr Ser
Trp Met Asn Trp Val Arg Gln Ala Pro Gly Lys Gly Leu Glu Trp Met
Gly Arg Ile Phe Pro Gly Asp Gly Asp Thr Asp Tyr Asn Gly Lys Phe
Lys Gly Arg Val Thr Ile Thr Ala Asp Lys Ser Thr Ser Thr Ala Tyr
Met Glu Leu Ser Ser Leu Arg Ser Glu Asp Thr Ala Val Tyr Tyr Cys
Ala Arg Asn Val Phe Asp Gly Tyr Trp Leu Val Tyr Trp Gly Gln Gly
Thr Leu Val Thr Val Ser Ser
amino acid sequences of variable region of the heavy chain (VH) of humanized
B-Ly1 antibody (B-HL15)
SEQ ID NO: 17
Glu Val Gln Leu Val Glu Ser Gly Gly Gly Leu Val Lys Pro Gly Ser
Ser Leu Arg Leu Ser Cys Ala Ala Ser Gly Phe Thr Phe Ser Tyr Ser
Trp Met Asn Trp Val Arg Gln Ala Pro Gly Lys Gly Leu Glu Trp Met
Gly Arg Ile Phe Pro Gly Asp Gly Asp Thr Asp Tyr Asn Gly Lys Phe
Lys Gly Arg Val Thr Ile Thr Ala Asp Lys Ser Thr Ser Thr Ala Tyr
Met Glu Leu Ser Ser Leu Arg Ser Glu Asp Thr Ala Val Tyr Tyr Cys
Ala Arg Asn Val Phe Asp Gly Tyr Trp Leu Val Tyr Trp Gly Gln Gly
Thr Leu Val Thr Val Ser Ser
amino acid sequences of variable region of the heavy chain (VH) of humanized
B-Ly1 antibody (B-HL16)
SEQ ID NO: 18
Glu Val Gln Leu Val Glu Ser Gly Gly Gly Leu Val Lys Pro Gly Gly
Ser Leu Arg Val Ser Cys Ala Ala Ser Gly Phe Thr Phe Ser Tyr Ser
Trp Met Asn Trp Val Arg Gln Ala Pro Gly Lys Gly Leu Glu Trp Met
Gly Arg Ile Phe Pro Gly Asp Gly Asp Thr Asp Tyr Asn Gly Lys Phe
Lys Gly Arg Val Thr Ile Thr Ala Asp Lys Ser Thr Ser Thr Ala Tyr
Met Glu Leu Ser Ser Leu Arg Ser Glu Asp Thr Ala Val Tyr Tyr Cys
Ala Arg Asn Val Phe Asp Gly Tyr Trp Leu Val Tyr Trp Gly Gln Gly
Thr Leu Val Thr Val Ser Ser
amino acid sequences of variable region of the heavy chain (VH) of humanized
B-Ly1 antibody (B-HL17)
SEQ ID NO: 19
Glu Val Gln Leu Val Glu Ser Gly Gly Gly Leu Val Lys Pro Gly Gly
Ser Leu Arg Leu Ser Cys Ala Ala Ser Gly Phe Thr Phe Ser Tyr Ser
Trp Met Asn Trp Val Arg Gln Ala Pro Gly Lys Gly Leu Glu Trp Met
Gly Arg Ile Phe Pro Gly Asp Gly Asp Thr Asp Tyr Asn Gly Lys Phe
Lys Gly Arg Val Thr Ile Thr Ala Asp Lys Ser Thr Ser Thr Ala Tyr
Met Glu Leu Ser Ser Leu Arg Ser Glu Asp Thr Ala Val Tyr Tyr Cys
Ala Arg Asn Val Phe Asp Gly Tyr Trp Leu Val Tyr Trp Gly Gln Gly
Thr Leu Val Thr Val Ser Ser
amino acid sequences of variable region of the light chain (VL) of humanized B-
Ly1 antibody B-KV1
SEQ ID NO: 20
Asp Ile Val Met Thr Gln Thr Pro Leu Ser Leu Pro Val Thr Pro Gly
Glu Pro Ala Ser Ile Ser Cys Arg Ser Ser Lys Ser Leu Leu His Ser
Asn Gly Ile Thr Tyr Leu Tyr Trp Tyr Leu Gln Lys Pro Gly Gln Ser
Pro Gln Leu Leu Ile Tyr Gln Met Ser Asn Leu Val Ser Gly Val Pro
Asp Arg Phe Ser Gly Ser Gly Ser Gly Thr Asp Phe Thr Leu Lys Ile
Ser Arg Val Glu Ala Glu Asp Val Gly Val Tyr Tyr Cys Ala Gln Asn
Leu Glu Leu Pro Tyr Thr Phe Gly Gly Gly Thr Lys Val Glu Ile Lys
Arg Thr Val

1-23. (canceled)

24. A method of treating diffuse large B-cell lymphoma (DLBCL) in a subject in need thereof, comprising administering to said subject a therapeutically effective amount of a BET inhibitor, a Bcl-2 inhibitor and an anti-CD20 antibody.

25. The method of claim 24, wherein the BET inhibitor is 2-[(S)-4-(4-chloro-phenyl)-2,3,9-trimethyl-6H-1-thia-5,7,8,9a-tetraaza-cyclopenta[e]azulen-6-yl]-N-[3-(4-methyl-piperazin-1-yl)-propyl]-acetamide.

26. The method of claim 25, wherein the BET inhibitor is administered subcutaneously at a dose between about 0.3 mg/kg/d and about 0.65 mg/kg/d for 14 consecutive days every 3 weeks.

27. The method of claim 24, wherein the Bcl-2 inhibitor is venetoclax.

28. The method of claim 27, wherein the Bcl-2 inhibitor is daily administered orally at a dose between about 400 mg/d and about 800 mg/d

29. The method of claim 24, wherein the anti-CD20 is a Type I anti-CD20 antibody, or a Type II anti-CD20 antibody wherein at least 40% of the N-linked oligosaccharides in the Fc region are non-fucosylated.

30. The method of claim 29, wherein the Type II anti-CD20 antibody is a humanized B-Ly1 antibody.

31. The method of claim 29, wherein the Type II anti-CD20 antibody is obinutuzumab.

32. The method of claim 29, wherein the Type I anti-CD20 antibody is rituximab.

33. The method of claim 29, wherein the anti-CD20 antibody is weekly administered intravenously at a dose of about 375 mg/m².

34. The method of claim 24, wherein the BET inhibitor is 2-[(S)-4-(4-chloro-phenyl)-2,3,9-trimethyl-6H-1-thia-5,7,8,9a-tetraaza-cyclopenta[e]azulen-6-yl]-N-[3-(4-methyl-piperazin-1-yl)-propyl]-acetamide, the Bcl-2 inhibitor is venetoclax, and the anti-CD20 antibody is rituximab.

35. The method of claim 24, wherein the BET inhibitor is co-administered with at least one of the Bcl-2 inhibitor and the anti-CD20 antibody.

36. The method of claim 24, wherein each of said BET inhibitor, Bcl-2 inhibitor and anti-CD20 antibody are administered separately.

37. The method of claim 24, said method further comprising administering a therapeutically effective amount of one or more additional other cytotoxic, chemotherapeutic or anti-cancer agents.

38. A pharmaceutical composition comprising a BET inhibitor and at least one of a Bcl-2 inhibitor and an anti-CD20 antibody; and one or more pharmaceutically acceptable excipients.

39. The pharmaceutical composition of claim 38, wherein the BET inhibitor is 2-[(S)-4-(4-chloro-phenyl)-2,3,9-trimethyl-6H-1-thia-5,7,8,9a-tetraaza-cyclopenta[e]azulen-6-yl]-N-[3-(4-methyl-piperazin-1-yl)-propyl]-acetamide.

40. The pharmaceutical composition of claim 38, wherein the Bcl-2 inhibitor is venetoclax.

41. The pharmaceutical composition of claim 38, wherein the anti-CD20 is a Type I anti-CD20 antibody, or a Type II anti-CD20 antibody wherein at least 40% of the N-linked oligosaccharides in the Fc region are non-fucosylated.

42. The pharmaceutical composition of claim 41, wherein the Type II anti-CD20 antibody is a humanized B-Ly1 antibody.

43. The pharmaceutical composition of claim 41, wherein the Type II anti-CD20 antibody is obinutuzumab.

44. The pharmaceutical composition of claim 41, wherein the Type I anti-CD20 antibody is rituximab.

45. The pharmaceutical composition of claim 38, wherein the BET inhibitor is 2-[(S)-4-(4-chloro-phenyl)-2,3,9-trimethyl-6H-1-thia-5,7,8,9a-tetraaza-cyclopenta[e]azulen-6-yl]-N-[3-(4-methyl-piperazin-1-yl)-propyl]-acetamide, the Bcl-2 inhibitor is venetoclax, and the anti-CD20 antibody is rituximab.

46. The pharmaceutical composition of claim 38, said composition further comprising one or more additional other cytotoxic, chemotherapeutic or anti-cancer agents.

47. A kit comprising a BET inhibitor, a Bcl-2 inhibitor and an anti-CD20 antibody for the simultaneous, separate or sequential administration of said BET inhibitor, Bcl-2 inhibitor and anti-CD20 antibody.

48. The kit of claim 47, wherein the BET inhibitor is 2-[(S)-4-(4-chloro-phenyl)-2,3,9-trimethyl-6H-1-thia-5,7,8,9a-tetraaza-cyclopenta[e]azulen-6-yl]-N-[3-(4-methyl-piperazin-1-yl)-propyl]-acetamide.

49. The kit of claim 47, wherein the BET inhibitor is 2-[(S)-4-(4-chloro-phenyl)-2,3,9-trimethyl-6H-1-thia-5,7,8,9a-tetraaza-cyclopenta[e]azulen-6-yl]-N-[3-(4-methyl-piperazin-1-yl)-propyl]-acetamide, the Bcl-2 inhibitor is venetoclax, and the anti-CD20 antibody is rituximab.

50. The kit of claim 47, said kit further comprising one or more additional other cytotoxic, chemotherapeutic or anti-cancer agents.
